(19) United States
(12) Patent Application Publication
(10) Pub. No.: US 2010/0305040 A1
(43) Pub. Date: Dec. 2, 2010

(54) PRODUCTION OF SOLUBLE RECOMBINANT PROTEIN BY PI VALUE CONTROL OF N-TERMINAL

(76) Inventors: Sang Jun Lee, Busan (KR); Young Ok Kim, Busan (KR); Bo Hye Nam, Busan (KR)

Correspondence Address: KLARQUIST SPARKMAN, LLP, 121 SW SALMON STREET, SUITE 1600, PORTLAND, OR, 97204, US

(21) Appl. No.: 12/745,187

(22) PCT Filed: Apr. 17, 2008
(86) PCT No.: PCT/KR2008/002173

§ 371 Date: May 27, 2010

(30) Foreign Application Priority Data
Nov. 28, 2007 (KR) 10-2007-0121977
Apr. 16, 2008 (KR) 10-2008-0035162

(51) Int. Cl. A61K 38/02; C12N 15/63; C12N 5/10; C12P 21/00; C12Q 1/02; C07K 19/00; C12P 21/06; A61P 25/00

(52) U.S. Cl. 514/17.7; 435/320.1; 435/325; 435/69.7; 435/29; 530/350; 435/68.1

(57) ABSTRACT

The present invention relates to a method for improving secretion efficiency of a recombinant foreign protein using a polypeptide fragment containing N-region of a signal sequence (directional signal) or variants thereof with the controlled pI value and/or a secretional enhancer composed of a hydrophilic polypeptide with the controlled pI value. The method of the present invention can be not only useful for the production of a recombinant foreign protein by preventing precipitation of an insoluble precipitate and by increasing extracellular or extra-periplasmic secretion efficiency of a recombinant protein but also useful for the transduction of an effective therapeutic protein by increasing membrane permeability using a strong secretional enhancer.

### TECHNICAL FIELD

The present invention relates to a method for improving secretion efficiency of a recombinant protein.

### BACKGROUND ART

One of the key techniques in modern bioengineering is the production of a recombinant protein. Particularly, the production of a water soluble protein in native form is important. The production of a water soluble protein is important for the production and recovery of an active protein, crystallization thereof for the functional studies and industrialization. Studies in relation to the production of a recombinant protein using E. coli have been undergoing. Using E. coli has many advantages such as easy manipulation, short culture time, safe expression, low costs and easy scale regulation.

Because the heterogenous recombinant protein produced in E. coli does not pass through post-translational chaperons or post-translational processing, there is no folding in the recombinant protein or it turns into an insoluble protein inclusion body (Baneyx, Curr. Opin Biotechnol. 10:411-421, 1999).

Since it was disclosed that signal sequence induces the extracellular secretion of a protein out of periplasm, studies on the structure and functions of signal sequence have been focused on amino terminal basic region (Lehnhardt, et al., J. Biol. Chem, 263:10300-10303, 1988), hydrophobic region (Goldstein et al., J. Bacteriol. 172:1225-1231, 1990) and cleavage region (Duffaud and Inouye, J. Biol. Chem, 263:10224-10228, 1988). At the same time, various vectors have been developed by using different signal sequences to produce a water soluble protein (ompA: Ghrayeb et al., EMBO J. 3:2437-2442, 1984; Duffaud et al., Methods Enzymol, 153:492-507, 1987; phoA: Dodt at al., FEBS Lett. 202:373-377, 1986; Kohl et al., Nucleic Acids Res. 18:1069, 1990; eltA: Morika-Fujimoto et al., J Biol Chem 266:1728-1732, 1991; bla: Oka et al., Agric Biol. Chem. 51:1099-1104, 1987; eltIIb-B: Jobling et al., Plasmid, 38:158-173, 1997). However, the vectors using signal sequence so far are limited in expressing a water soluble protein and even the expressed protein as a recombinant fusion protein form, which contains the cleavage site of signal peptidase or protease at the N-terminal after cleaving, so that it is very difficult to obtain a recombinant protein having the native amino terminal. The reasons which make the production of a recombinant protein using signal sequence difficult are 1) it is impossible to predict the production of a water soluble protein and many researchers believe that water-solubility of a recombinant protein depends on the characteristics of the amino acid sequence of a whole protein; and 2) there are so many different sequences acting as signal sequence and there is no proper analysis method to investigate the interaction for SecA/signal peptides, yet (Triplett et al., J Biot Chem 276:19648-19655, 2001).

The present inventors provided the expression vector containing the gene construct composed of polynucleotides each encoding the truncated signal sequence containing N-region and/or N-region harboring hydrophobic fragment as directional signal and/or the truncated signal sequence harboring secretional enhancer composed of hydrophilic polypeptides, which is described in Korean Patent Publication No: 10-2007-0009453, and also confirmed previously that the soluble expression of an adhesive protein Mefp1 could be improved by adding the nucleotide encoding the adhesive protein Mefp1 to the vector containing the above gene construct. The present inventors also analyzed the pI values depending on the length of N-region fragment of a signal sequence and confirmed that it was important for the fragments, which are from OmpASP₁₋₃, to the full length of OmpASP₁₋₂₁ to have the equal pI value (10.55) for the expression of an adhesive protein Mefp1. In Korean Patent Publication No: 10-2007-0009453, the present inventors reported that the protein containing amphiphilic domain such as olive flounder Hepcidin I is limited in the soluble expression, with using the N-region fragment of the signal sequence alone, and further established a method for improving the expression of olive flounder Hepcidin I as a water soluble form by adding a hydrophilic secretional enhancer sequence to the gene construct.

Thus, the present inventors constructed a recombinant expression vector containing the gene construct composed of polynucleotides encoding signal sequences having various pI values, confirmed that the pI value of the N-terminal of the signal sequence included in the recombinant vector played a certain role in the soluble expression of a foreign protein and proved the interrelation between the pI value of the N-terminal of the signal sequence and the pI value of the secretional enhancer when the secretional enhancer was necessary because of the structural characteristics of the foreign protein, and further confirmed that the control of pI value of the N-terminal of the signal sequence in the recombinant expression vector constructed for the expression of a foreign protein was important in improvement of the soluble expression of the foreign protein, leading to the completion of this invention.

### DISCLOSURE

### Technical Problem

It is an object of the present invention to provide an effective method for producing a water soluble recombinant fusion protein for a foreign gene and recovering the protein having the native form at amino terminal.

### Technical Solution

To achieve the above object, the present invention provides an expression vector for improving secretion efficiency of a foreign protein containing a gene construct which comprises (i) a promoter and (ii) a polynucleotide operably linked to the promoter encoding a polypeptide fragment containing a signal sequence and/or pI value of the N-region of the leader sequence of a foreign protein and/or N-region of the leader sequence or variants thereof in which the distance between amino acids affecting the pI value is controlled.

The present invention also provides an expression vector for improving secretion efficiency of a foreign protein containing a gene construct which comprises (i) a promoter; (ii) a polynucleotide operably linked to the promoter encoding a polypeptide fragment containing signal sequence and/or N-region of the leader sequence or variants thereof in which the pI value is controlled; and (iii) a polynucleotide operably linked to the polynucleotide encoding the polypeptide fragment or variants thereof encoding a secretional enhancer comprising a hydrophilicity enhancing sequence with the controlled pI value.

The present invention also provides a transformant prepared by transforming a host cell with the above expression vector.

The present invention also provides a method for improving secretion efficiency of a recombinant protein using the transformant.

The present invention also provides a method for producing a recombinant fusion foreign protein.

The present invention also provides a recombinant fusion foreign protein produced by the above method.

The present invention also provides a pharmaceutical composition containing the above recombinant fusion foreign protein and a pharmaceutically acceptable carrier.

The present invention also provides a method for producing a foreign protein in the native form.

In addition, the present invention provides a method for producing an intracellular carrier of a target material.

### Advantageous Effect

The method of the present invention favors the prevention of a recombinant protein from being precipitated as an insoluble protein and the improvement of secretion efficiency of the protein out of cytoplasm or into periplasm, so that it can be effectively used for the production of a recombinant foreign protein and for the transduction of a therapeutic protein by increasing membrane permeability using a strong secretional enhancer.

### DESCRIPTION OF DRAWINGS

The application of the preferred embodiments of the present invention is best understood with reference to the accompanying drawings, wherein:

FIG. 1 is a diagram illustrating the comparative soluble expressions of the adhesive protein Mefp1 (soluble fraction: approximately 20 μg) by the signal sequence OmpASPₜᵣ and its variant leader sequences (arrow: recombinant Mefp1). Values obtained from densitometer analysis present the comparative mean values of the expressions of the protein in three different clones:

(A) SDS-PAGE;

(B) Western blotting; and,

(C) densitometer analysis;

1a:

M: marker;

(SEQ. ID. NO: 15)

line 1: Met-Ala-Lys(pI 9.90);

(SEQ. ID. NO: 16)

line 2: Met-Lys-Ala-Lys(pI 10.55);

(SEQ. ID. NO: 17)

line 3: Met-Lys-Lys-Ala-Lys(pI 10.82);

(SEQ. ID. NO: 18)

line 4: Met-Lys-Lys-Lys-Ala-Lys(pI 10.99);

(SEQ. ID. NO: 19)

line 5: Met-Lys-Lys-Lys-Lys-Ala-Lys(pI 11.11);

(SEQ. ID. NO: 20)

line 6: Met-Lys-Lys-Lys-Lys-Lys-Ala-Lys(pI
11.21);

(SEQ. ID. NO: 21)

line 7: Met-Lys-Lys-Lys-Lys-Lys-Lys-Ala-Lys(pI
11.28);
and,

(SEQ. ID. NO: 22)

line 8: Met-Lys-Lys-Lys-Lys-Lys-Lys-Lys-Lys-Ala-
Lys(pI 11.41);

1b:

M: marker;

(SEQ. ID. NO: 15)

line 1: Met-Ala-Lys (pI 9.90);

(SEQ. ID. NO: 23)

line 2: Met-Arg-Ala-Lys (pI 11.52);

(SEQ. ID. NO: 24)

line 3: Met-Arg-Arg-Ala-Lys (pI 12.51);

(SEQ. ID. NO: 25)

line 4: Met-Arg-Arg-Arg-Arg-Ala-Lys (pI 12.98);

(SEQ. ID. NO: 26)

line 5: Met-Arg-Arg-Arg-Arg-Arg-Arg-Ala-Lys (pI
13.20);
and,

(SEQ. ID. NO: 27)

line 6: Met-Arg-Arg-Arg-Arg-Arg-Arg-Arg-Arg-Ala-
Lys (pI 13.35).

FIG. 2 is a diagram illustrating the comparative soluble expressions of the adhesive protein Mefp1 (soluble fraction: approximately 20 μg) by the clones modified in the leader sequence (Met-Ala-Lys) of the recombinant vector pET22b(+)(ompASP₁-7×mefp1*) (arrow: recombinant Mefp1). Values obtained from densitometer analysis present the comparative mean values of the expressions of the protein in three different clones:

(A) SDS-PAGE;

(B) Western blotting; and,

(C) densitometer analysis;

M: marker;

(SEQ. ID. NO: 35)

line 1: Met-Asp-Asp-Asp-Asp-Asp-Ala-Ala(pI 2.73);

(SEQ. ID. NO: 36)

line 2: Met-Asp-Asp-Asp-Ala-Ala(pI 2.87);

(SEQ. ID. NO: 37)

line 3: Met-Glu-Glu(pI 3.09);

(SEQ. ID. NO: 38)

line 4: Met-Ala-Glu(pI 3.25);

(SEQ. ID. NO: 39)

line 5: Met-Ala-Ala(pI 5.60);

(SEQ. ID. NO: 40)

line 6: Met-Cys-His(pI 7.13);

(SEQ. ID. NO: 41)

line 7: Met-Ala-His(pI 7.65);

(SEQ. ID. NO: 15)

line 8: Met-Ala-Lys(pI 9.90);
and,

(SEQ. ID. NO: 25)

line 9: Met-Arg-Arg-Arg-Arg-Ala-Lys(pI 12.98).

FIG. 3 is a diagram illustrating the soluble expression of the recombinant Mefp1 fusion protein (soluble fraction: approximately 20 μg) obtained from the clones having different distances between the leader sequence region Met-Glu-Glu (pI 3.09) and factor Xa recognition site (Xa) (arrow: recombinant Mefp1):

(A) SDS-PAGE; and,

(B) Western blotting;

M: marker;

(SEQ. ID. NO: 37)

line 1: Met-Glu-Glu(pI 3.09);

(SEQ. ID. NO: 46)

line 2: Met-Glu-Glu-Xa(pI 4.01);

(SEQ. ID. NO: 47)

line 3: Met-Glu-Glu-Pro-Ser-Xa(pI 4.01);

(SEQ. ID. NO: 48)

line 4: Met-Glu-Glu-Pro-Ser-Tyr-Pro-Xa(pI 4.01);
and,

(SEQ. ID. NO: 49)

line 5: Met-Glu-Glu-Pro-Ser-Tyr-Pro-Pro-Thr-Xa(pI
4.01).

FIG. 4 is a diagram illustrating the soluble expression of the recombinant Mefp1 fusion protein (soluble fraction: approximately 20 μg) obtained from the leader sequence clones designed by modifying the pET-22b(+)[ompASP₁₋₁₁-7×mefp1*](*: Ra-6× His) clone to have different lengths in between Lys-Lys (arrow: recombinant Mefp1):

(A) SDS-PAGE; and,

(B) Western blotting;

4a:

M: marker;

(SEQ. ID. NO: 56)

line 1: Met-Lys-Lys-Thr-Ala-Ile-Ala-Ile-Ala-Val-
Ala-Ala-Lys(pI 10.82);

(SEQ. ID. NO: 57)

line 2: Met-Lys-Lys-Thr-Ala-Ile-Ala-Ile-Ala-Val-
Ala-Ala-Ala(pI 10.55, d₁ = 0);

(SEQ. ID. NO: 58)

line 3: Met-Lys-Ala-Thr-Lys-Ile-Ala-Ile-Ala-Val-
Ala-Ala-Ala(pI 10.55, d₁ = 2);

(SEQ. ID. NO: 59)

line 4: Met-Lys-Ala-Thr-Ala-Ile-Lys-Ile-Ala-Val-
Ala-Ala-Ala(pI 10.55, d₁ = 4);

(SEQ. ID. NO: 60)

line 5: Met-Lys-Ala-Thr-Ala-Ile-Ala-Ile-Lys-Val-
Ala-Ala-Ala(pI 10.55, d₁ = 6);
and,

(SEQ. ID. NO: 61)

line 5: Met-Lys-Ala-Thr-Ala-Ile-Ala-Ile-Ala-Val-
Lys-Ala-Ala(pI 10.55, d₁ = 8);

4b :

M: marker;

(SEQ. ID. NO: 17)

line 1: Met-Lys-Lys-Ala-Lys(pI 10.82);

(SEQ. ID. NO: 106)

line 2: Met-Lys-Ala-Thr-Ala-Ile-Lys-Ala-Lys
(pI 10.82, d₁ = 4, d₂ = 1);

(SEQ. ID. NO: 107)

line 3: Met-Lys-Ala-Thr-Ala-Ile-Lys-Ala-Ala-Lys(pI
10.82, d₁ = 4, d₂ = 2);

(SEQ. ID. NO: 108)

line 4: Met-Lys-Ala-Thr-Ala-Ile-Lys-Ala-Ala-Ala-
Lys(pI 10.82, d₁ = 4, d₂ = 3);
and,

(SEQ. ID. NO: 109)

line 5: Met-Lys-Ala-Thr-Ala-Ile-Lys-Ala-Ala-Ala-
Ala-Lys(pI 10.82, d₁ = 4, d₂ = 4).

FIG. 5 is a diagram illustrating the effect of the amino acid sequence pI value and hydrophobic value of Met-7 x homologous amino acids inserted as a leader sequence to give signaling function and secretion enhancing function on the ofHepcidin I soluble expression (soluble fraction: approximately 20 μg) (arrow: recombinant ofHepcidin I):

(A) SDS-PAGE; and,

(B) Western blotting;

M: marker;

(SEQ. ID. NO: 70)

line 1: MRRRRRRR(pI 13.28, hy +1.97);

(SEQ. ID. NO: 71)

line 2: MKKKKKKK(pI 11.28, hy +1.97);

(SEQ. ID. NO: 72)

line 3: MHHHHHHH(pI 8.08, hy −0.35);

(SEQ. ID. NO: 73)

line 4: MYYYYYYY(pI 5.59, hy −1.55);

(SEQ. ID. NO: 74)

line 5: MCCCCCCC(pI 4.57, hy −0.69);

(SEQ. ID. NO: 75)

line 6: MEEEEEEE(pI 2.78, hy +1.97);
and,

(SEQ. ID. NO: 76)

line 7: MDDDDDDD(pI 2.52, hy +1.97).

FIG. 6 is a diagram illustrating the effect of N-terminal pI value in the leader sequence composed of OmpASP fragment(Met-2 aas)-OmpASP₄₋₁₀-secretional enhancer candidate sequence-Xa with the controlled pI value on the secretional enhancer sequence and the soluble expression (soluble fraction: approximately 20 μg) (arrow: recombinant ofHepcidin I):

(A) SDS-PAGE; and,

(B) Western blotting;

M: marker;

(SEQ. ID. NO: 86)

line 1: MAH(pI 7.65)-OmpASP₄₋₁₀(pI 5.70)-6x Arg
(pI 13.20; hy +1.75)-Xa(pI 7.05);

(SEQ. ID. NO: 87)

line 2: MAH-OmpASP₄₋₁₀-6x Tyr(pI 5.55; hy −1.33)-
Xa;

(SEQ. ID. NO: 88)

line 3: MAH-OmpASP₄₋₁₀-6x Glu(pI 2.82; hy +1.75)-
Xa;

(SEQ. ID. NO: 89)

line 4: MAA(pI 5.60)-OmpASP₄₋₁₀-6x Arg-Xa;

(SEQ. ID. NO: 90)

line 5: MAA-OmpASP₄₋₁₀-6x Tyr-Xa;

(SEQ. ID. NO: 91)

line 6: MAA-OmpASP₄₋₁₀-6x Glu-Xa;

(SEQ. ID. NO: 92)

line 7: MEE(pI 3.09)-OmpASP₄₋₁₀-6x Arg-Xa;

(SEQ. ID. NO: 93)

line 8: MEE-OmpASP₄₋₁₀-6x Tyr-Xa;
and,

(SEQ. ID. NO: 94)

line 9: MEE-OmpASP₄₋₁₀-6x Glu-Xa.

### BEST MODE

The terms used in this invention are described hereinafter.

“Heterologous protein” or “target heterologous protein” is the protein targeted by those in the art for mass-production, which includes every protein that is possibly expressed in a transformant using a recombinant expression vector containing a polynucleotide encoding the protein.

“Fusion protein” indicates the protein produced with the addition of another amino acid sequence or with fusion of another protein to N-terminal or C-terminal of the original foreign protein.

“Signal sequence” is an effective sequence that helps a foreign protein expressed in virus, prokaryotic cells or eukaryotic cells efficiently pass through intracellular membrane for extracellular or extra-periplasmic secretion. The signal sequence is composed of positively charged N-region, central characteristic hydrophobic region and C-region with a cleavage site. The signal sequence fragment used in this invention indicates the full length or a part of the sequence containing positively charged region, central characteristic hydrophobic region and C-terminal with a cleavage site.

“Leader sequence” indicates the amino acid sequence of N-terminal of a foreign protein.

“Polypeptide fragment” indicates the polypeptide sequence having a specific polypeptide function and minimum length or a bigger size. Unless stated otherwise, the full length polypeptide is not included in the “polypeptide fragment” of the invention. For example, ‘the polypeptide fragment containing N-region of a signal sequence’ indicates the shortened signal sequence functioning as a signal sequence and the entire signal sequence is not included.

“Polynucleotide” indicates a polymer molecule wherein two or more nucleic acid molecules are linked by phosphodiester bond, which includes DNA and RNA.

“Secretional enhancer” indicates a hydrophilic polypeptide composed of hydrophilic amino acids, which plays a role in increasing hydrophilicity behind the signal sequence or the leader sequence.

“N-region of a signal sequence” is a part preserved in the general signal sequence, which is a strong basic sequence, located at the N-terminal and comprising 1-10 amino acids according to the signal sequence.

“Central specific hydrophobic region” indicates the region following N-region in the general signal sequence, which shows strong hydrophobicity owing to many hydrophobic amino acids.

“Signal sequence fragment” indicates a part of a signal sequence, and unless stated otherwise, it is a fragment of a signal sequence with the deletion of C-terminal.

“Signal sequence fragment variant” indicates the fragment prepared by changing any sequence region except the first amino acid Met in a signal sequence.

“protease recognition site” indicates a specific amino acid sequence region cleaved by a protease.

“Amphipathic domain” is the domain having both hydrophilic and hydrophobic regions, which is an inside region of a protein having a similar structure with transmembrane domain. In this invention, it is identical to the “transmembrane-like domain” in its meaning.

“Transmembrane-like (TM-like) domain” indicates the region expected to have a similar structure with transmembrane domain of a transmembrane protein in polypeptide amino acid sequence analysis (Brasseur et al., Biochim Biophys Acta 1029(2):267-273, 1990). In general, the transmembrane-like domain is easily predicted by various computer softwares predicting transmembrane domain. And the softwares are exemplified by TMpred, HMMTOP, TBBpred, DAS-TMfilter (//www.enzim.hu/DAS/DAS.html), etc. The “transmembrane-like domain” herein includes “transmembrane domain” confirmed to have real transmembrane characteristics.

“Expression vector” is a linear or a circular DNA molecule composed of a fragment encoding a target polypeptide operably linked to an additional fragment for the transcription of the vector. The additional fragment includes a promoter and a stop codon sequence. The expression vector contains one or more origins, one or more selection markers, an enhancer, a polyadenylation signal and others. The expression vector is generally originated from plasmid or virus DNA or contains elements from the both.

“Operably linked” means that fragments are arranged to be functioning as they are supposed to be, for example once transcription starts at the promoter, it goes through coded fragment to stop codon.

Hereinafter, the present invention is described in detail.

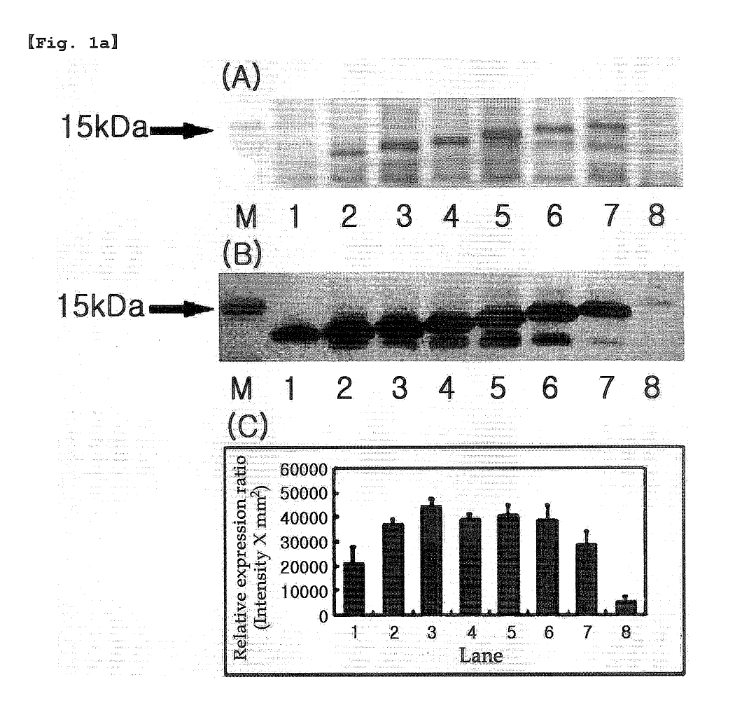

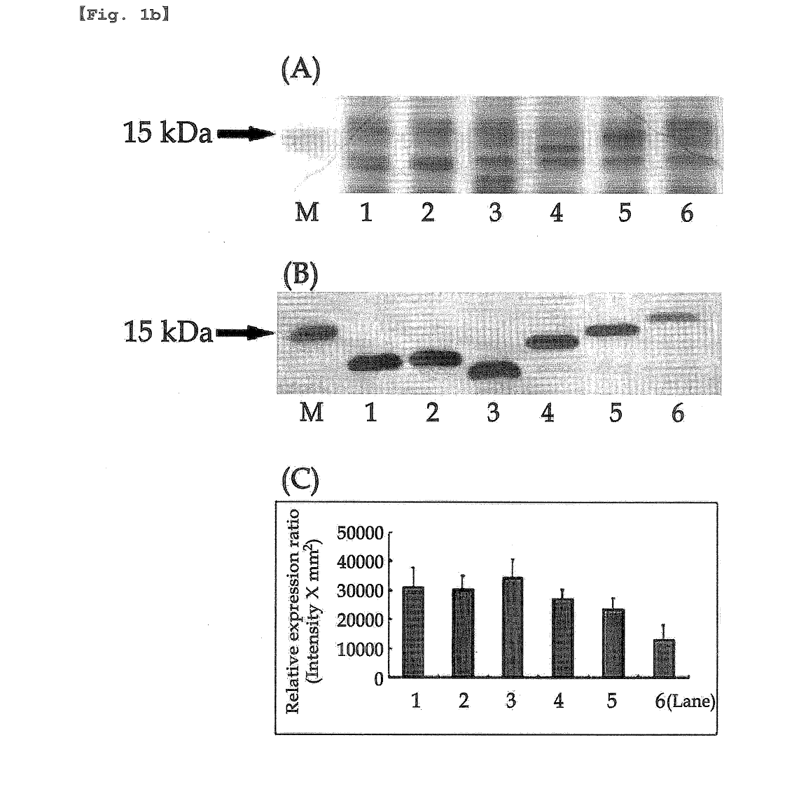

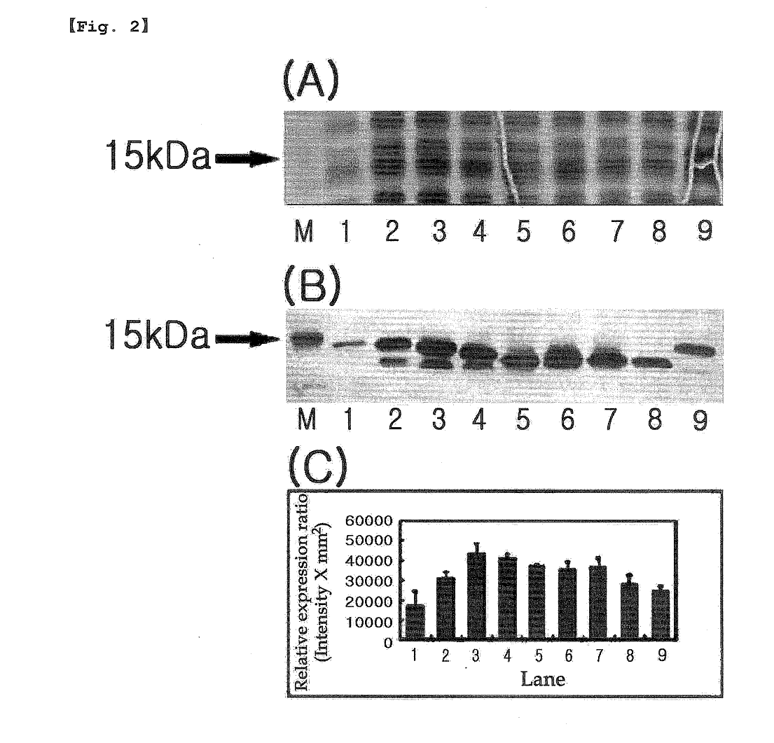

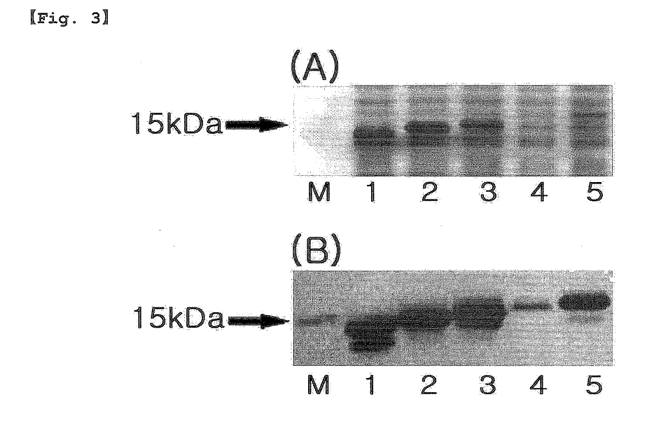

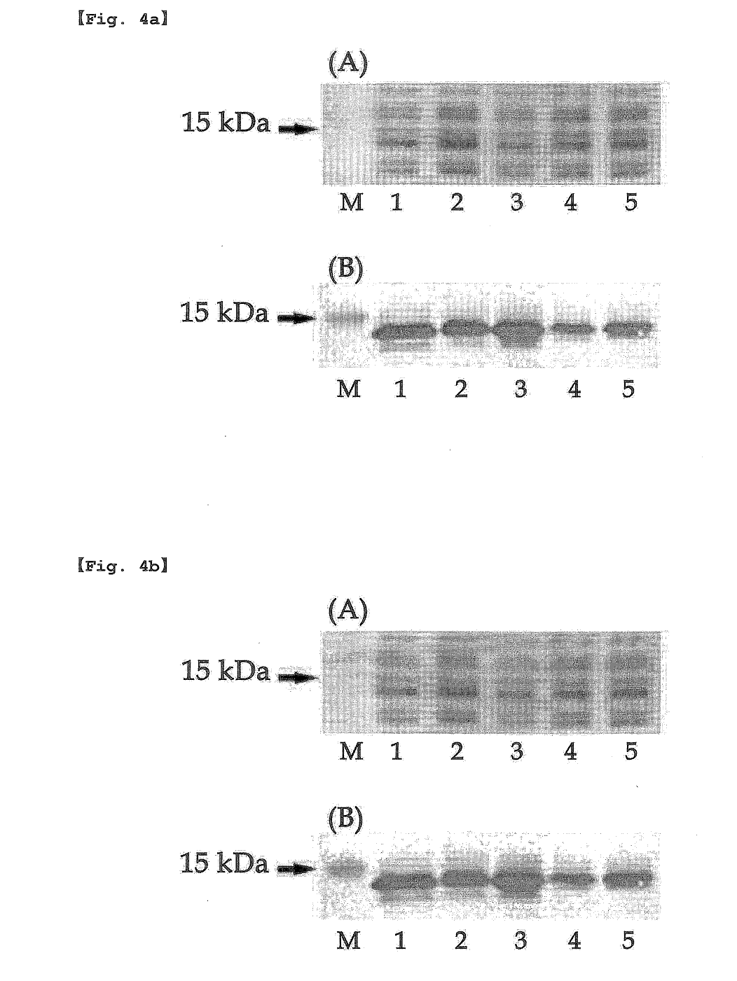

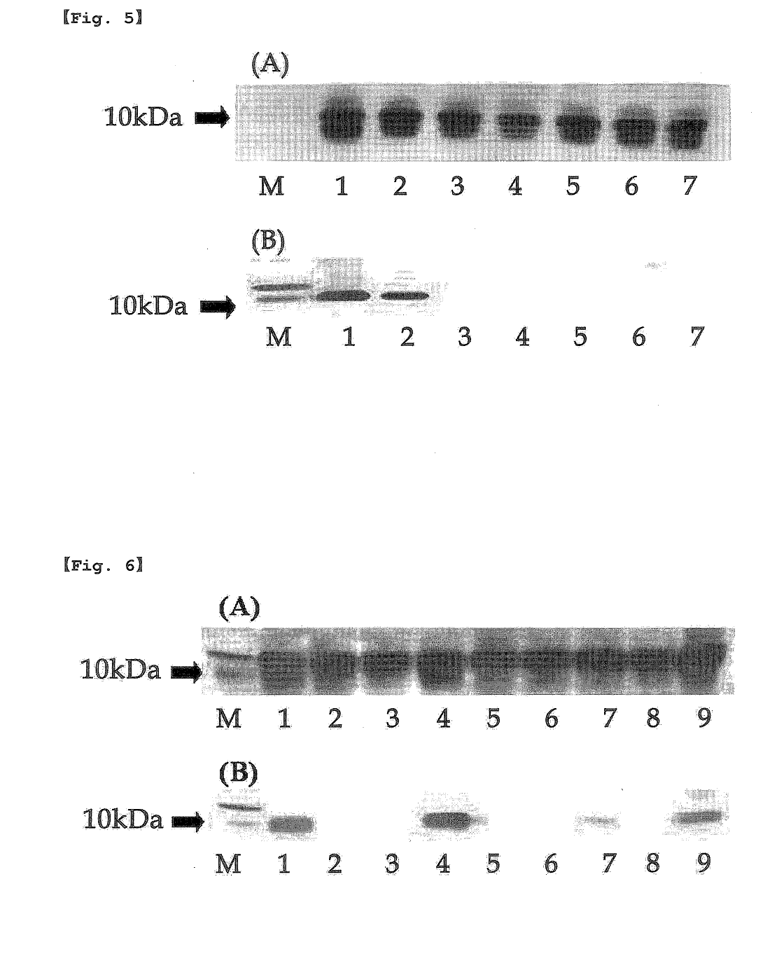

The present inventors constructed pET-22b(+)(ompASP₁-7×mefp1*) and pET-22b(+)(ompASP₁₋₂-7×mefp1*) clones by the fusion of the coding sequences of OmpASP₁(Met) and OmpASP₁₋₂(Met-Lys), which are parts of OmpA signal peptide (OmpASP) that is the signal sequence inducing protein secretion in E. coli, to 5′ end of 7×mefp1 encoding an adhesive protein Mefp1 (see Table 1). E. coli BL21 (DE3) was transformed with the constructed clone vectors, followed by expression. As a result, the change of the only one amino acid (Lysine; Lys; K; pI=9.72) made significant difference in soluble expressions of the proteins Met-7×Mefp1* (SEQ. ID. NO: 15) and Met-Lys-7×Mefp1* (SEQ. ID. NO: 16) from the above two clones (see FIG. 1a, line 1 and line 2). So, it was confirmed that Lys that affects the pI value at the N-terminal played an important role in the soluble expression. Then, the sequence ranging from the Met end of OmpASPₜᵣ to the second last amino acid Lys (Ala-Lys) of N-terminal was determined as a standard for calculating the pI value of the leader sequence. The pI values from the OmpASP fragment (Met[M] or Met-Lys) to the first two amino acids (Ala-Lys) of the Mefp1 proteins were analyzed by using the computer program DNASIS™ (Hitachi, Japan). As a result, the pI value of Met-Ala-Lys was 9.90 and the pI value of Met-Lys-Ala-Lys was 10.55 (see Table 1). To confirm the above results, pET-22b(+)(ompASP₁₋₃-7×mefp1*) clone was constructed by the fusion of the coding sequence of the signal sequence fragment OmpASP₁₋₃ (Met-Lys-Lys) (SEQ. ID. NO: 17) having additional Lys, compared with OmpASP₁₋₂ , followed by investigation of the soluble expression by the same manner as described above. As a result, the pI value from the OmpASP₁₋₃ to the first two amino acids (Ala-Lys) in the leader sequence, which was Met-Lys-Lys-Ala-Lys, was 10.82, which supports the good relation with the increase of the soluble expression (see FIG. 1a, line 3).

To confirm whether or not the control of pI value could affect the soluble protein expression, pET-22b(+)(ompASP₁₋₃-Lysₙ-7×mefp1*) clone was constructed by inserting Lys in between the OmpASP₁₋₃ fragment and the first amino acid Ala of Mefp1 to increase pI value. And pET-22b(+)(ompASP₁-Argₙ-7×mefp1*) clone was also constructed by inserting Arg in between Met(OmpASP₁) and the first amino acid Ala of Mepf1 to increase pI value. The pI values from the N-terminal to the first two amino acids (Ala-Lys) of Mefp1 of the above proteins were analyzed (see Table 1). E. coli BL21 (DE3) was transformed with the constructed clone vectors, followed by expression. The expression was compared with the expressions of the proteins Met-7×Mefp1* (SEQ. ID. NO: 15), Met-Lys-7×Mefp1* (SEQ. ID. NO: 16) and Met-Lys-Lys-7×Mefp1* (SEQ. ID. NO: 17). As a result, the soluble expression of the protein in which the pI value was increased by the additional Lys to 10.99-11.21 (SEQ. ID. NO: 18- NO: 20) was similar to the control with the pI value of 10.55 (SEQ. ID. NO: 16) (see FIG. 1, line 4-line 6), but the reducing expression started from the pI value of 11.28 (see FIG. 1, line 7). Particularly, when the pI value was 11.41 (SEQ. ID. NO: 22), the expression was significantly reduced (see FIG. 1, line 8). The above results indicate that the pI value is specifically involved in membrane permeability when the pI value of the leader sequence is 10.55 or up. When the pI value of the leader sequence is increased by Lys to 10.82-11.41, that is the leader sequence has additional Lys which is OmpASP₁₋₃-Lysₙ-Ala-Lys, the sequence is expected to have equal transmembrane channel to OmpASP with the pI value of 10.55.

The soluble expressions of the proteins with the pI values of 11.52-13.35 (SEQ. ID. NO: 23- NO: 27) increased by addition of Arg were reduced as the pI value increased, except that the expression was slightly increased when the pI value of the leader sequence linked to two Args was 12.51. And the expression was significantly reduced when the pI value was 13.35 (see Table 1 and FIG. 1b). In the case of Arg, the reduced molecular weight of the region where two Args were linked (see FIG. 1b, line 3) was presumably resulted from the cleaving of a part of the leader sequence by protease. So, it was presumed that the leader sequences containing additional Arg in their sequence OmpASP₁-Argₙ-Ala-Lys commonly had Arg specific membrane permeability. However, interrelation between Arg specific membrane permeability mechanism and the signal sequence of TAT (twin-arginine translocation) system (Berks, Mol. Microbiol. 22:393-404, 1966) is not explained herein.

To investigate the effect of the N-terminal of the leader sequence with the low-controlled pI value on the soluble expression of a target protein, pET-22b(+)(ompASP₁-7×mefp1*) was used as the control and Ala-Lys of the leader sequence Met-Ala-Lys were differently modified to regulate the pI values to 2.73-7.65 (SEQ. ID. NO: 35- NO: 41), resulting in the construction of the leader sequence variants (MDDDDDAA; pI2.73, MDDDAA; pI2.87, MEE; pI3.09, MAE; pI3.25, MAA; pI5.60, MCH; pI7.13, MAH; pI7.65) (see Table 2), followed by investigation of their expressions. As a result, the soluble expression of the adhesive protein Mefp1 was similar or higher than that of the control with the pI value of 9.90, when the pI values were 2.87-7.65. In particular, the expression was the highest when the pI value was 3.09 (SEQ. ID. NO: 37) (see FIG. 2, line 3). The expression pattern was as follows: There were two kinds of expressions (Asp/Glu specific expression at the pI value 2.73-3.25 and moderate increase of the expression at the pI value of 3.25-9.90). So, it was confirmed that there were two different spectrums in the soluble expression of the adhesive protein Mefp1 induced by the pI value of N-terminal in the leader sequence with down-controlled pI. That is, the pI value control of N-terminal affects the soluble protein expression. The leader sequence N-terminal variants, MAA(pI5.60)(SEQ. ID. NO: 39), MCH(pI7.13) (SEQ. ID. NO: 40) and MAH(pI7.65) (SEQ. ID. NO: 41), are the sequences having weak interrelation with electric charge. In that short sequence of the variants, it is unlikely that a secretional enhancer is located which is described in Korean Patent Publication No. 10-2007-0009453. Therefore, the expression is regulated by the pI value of the leader sequence N-terminal variant. As described hereinbefore, the expressions of the adhesive protein Mefp1 having high+charge in the leader sequence expressed from pET-22b (+) (ompASP₁₋₃-Lysₙ-7×mefp1*) or pET-22b (+) (ompASP₁-Argₙ-7×mefp1*) and the adhesive protein Mefp1 having strong—charge in the leader sequence MDDDDDAA(SEQ. ID. NO: 35) were not related with electric charge.

Based on the above results, the pI-dependent soluble expression patterns were investigated. In the case of the soluble expression of the adhesive protein Mefp1 at high pI, \textcircled{1} Lys specific membrane permeation mechanism is involved at around the pI value of 10.82 (9.90-11.41), and \textcircled{2} Arg specific membrane permeation mechanism is involved at around the pI value of 12.51 wherein have the Arg (11.52-13.35). In the wide low range of pI (2.73-9.90), \textcircled{3} Asp/Glu specific membrane permeation mechanism is involved at the low pI value (2.73-3.25) and \textcircled{4} comparatively non-specific membrane permeation mechanism is involved at the pI value of 3.25-9.90. Accordingly, it is presumed that the leader sequence with the high pI value has Lys specific OmpASP Sec system (pI 9.90-11.41) and Arg specific membrane permeation mechanism (pI 11.52-13.35), the leader sequence with the wide low range of pI (pI 2.73-9.90) has Asp/Glu specific membrane permeation mechanism (pI 2.73-3.25, optimum pI: 3.09) and has comparatively non-specific membrane permeation, a kind of passive membrane permeation mechanism without the central point pI value in the range of 3.25-9.90. The above four membrane permeation mechanisms had no relationship with the expression and the increase of electric charge. So, the result of the analysis of interrelation between the pI value and the membrane permeability of a protein can be effectively used for the further studies on the expression of a soluble recombinant protein based on the membrane permeation mechanism.

The leader sequences exhibiting low expression rates at the high pI value of 11.41 and 13.35 (SEQ. ID. NO: 22 and SEQ. ID. NO: 27) had comparatively high hydrophilic value of 1.93, and the leader sequence (SEQ. ID. NO: 35) exhibiting low expression rate at the low pI value of 2.73 also had comparatively high hydrophilic value of 1.09. The significantly increased hydrophilicity in the leader sequence might result in the decrease of membrane permeability by inducing the binding of the hydrophilic transmembrane like domain with the lipid bilayer membrane, which is consistent with the hypothesis proposed in the earlier patent application (Korean Patent Publication No. 10-2007-0009453) saying that the hydrophilic transmembrane like domain inhibits the soluble expression of olive flounder Hepcidin I. However, when Lys is added, hydrophilicity of the leader sequence (SEQ. ID. NO: 18-21) can be offset to some degree, even though the leader sequence still has high hydrophilicity. So, It is very interesting that the addition of Lys leads to the increase of membrane permeability.

From the investigation of the expression increased by MEE(pI 3.09)(SEQ. ID. NO: 37), one of the optimum pI for the leader sequence of the adhesive foreign protein Mefp1 was judged to be 3.09. To optimize the distance between the leader sequence variant and a foreign protein linked thereto and to produce a protein having the native amino terminal, factor Xa recognition site (Xa) was inserted into the sequence, resulting in MEE(i=n)-Xa, and the amino acid of OmpASP₄₋₉ not affecting the pI value was inserted into the ( )as insert(i)=n(0, 2, 4, 6). As a result, pET-22b(+)(MEE-(i=n)-Xa-7×mefp1*) was constructed (see Table 3). When no amino acids were inserted in between MEE and Xa, and when two amino acids were inserted therein, the expression of a soluble protein was the highest (see FIG. 3). That is, the distance between the leader sequence and the factor Xa recognition site was the optimum when i=0-2. The soluble protein herein contains a factor Xa recognition site, so that it can be produced as a recombinant protein having the native form of N-terminal by treating factor Xa protease according to the conventional method known to those in the art.

After confirming that the pI value of N-terminal of signal sequence could affect the soluble expression of the adhesive protein Mefp1, the present inventors tried to confirm whether or not the distance of pI affecting amino acids (for example Lys) could be an element affecting the soluble expression of the protein. In N-terminal of a protein, the leader sequences MKAK and MKK have same pI values, but when two Lys-Lys are distant because of the insertion of a none pI specific amino acid such as Ala (Alanine; A) in between Lys-Lys, there might be difference in functions. So, based on the amino acid sequence of the signal sequence OmpASP fragment, MK₁-(d=n)-K₂-(8-n) was constructed and amino acids of OmpASP₁₋₁₁ not affecting the pI value were inserted into ( ) as d₁=n(0, 2, 4, 6, 8), resulting in the construction of pET⁻22b(+)[MK₁-(d₁=n)-K₂-(8-n)-AA-mefp1₃₋₁₀-6×mefp1*] clone (see Table 4). As a result, when d₁=4, indicating the distance between two Ks (d₁=distance of K₁-K₂) was 4, the expression of the soluble protein was most significant (see Table 4 and FIG. 4a). That is, the distance of amino acids was optimized when d₁=4. Additionally, when d₁=4, Ala (underlined part) of the clone was substituted with Lys(K₃) and Ala (d₂=n(1, 2, 3, 4) was inserted in between K₂ and K₃, resulting in the construction of pET-22b(+)[MK₁-(d₁=4)-K₂-(d₂=n)-AK₃-mefp1₃₋₁₀-6×mefp1*] (see Table 4). As a result, the optimum distance between two Ks (d₂=distance of K₂-K₃) was d₂=2>1>4>3. These results indicate that the distance is directly related to the soluble expression of an adhesive protein Mefp1. The above results also suggest that the important factor in the expression is not the sequence but the pI value and the distance between Lys-Lys in the leader sequence (see Table 4 and FIG. 4b).

As shown in FIG. 1a line 1, the adhesive protein Mefp1 is the protein that is able to be soluble-expressed by attaching only Met of a signal sequence to N-terminal of the protein. The soluble expression of the adhesive protein Mefp1 can be increased by regulating the pI values of the signal sequence and the leader sequence, and the distance between the pI specific amino acids. Olive flounder Hepcidin I protein contains amphipathic domain or transmembrane-like domain. According to Korean Patent Publication No. 10-2007-0009453, this protein could be soluble-expressed only when a secretional enhancer having signal sequence functions and hydrophilicity high enough to offset the internal TM-like domain was added. To soluble-express the olive flounder Hepcidin I protein, the present inventors designed the leader sequence of N-terminal as M-7×homologous amino acids in order to be functioning as a signal sequence and at the same time a secretional enhancer, and then constructed pET-22b(+)(ompASP₁-7×homologous amino acids-ofhep I**) for the expression of the protein having the controlled pI value of 2.52-13.28 and hydrophobicity of −1.55-+1.97 (see Table 5). The homologous amino acid herein was selected from the group consisting of arginine (Arg; R), lysine (Lys, K), histidine (His; H), tyrosine (Tyr; Y), cysteine (Cys; C), glutamic acid (Glu; E) and aspartic acid (Asp; D), which was supposed to have 7 repeats. The hydrophobicity was measured by DNASIS™ (Hitachi, Japan) as Hopp & Woods scale (window size: 6, threshold: 0.00). If the hydrophobicity value is +, it means the peptide is hydrophilic, while if the hydrophobicity value is −, the peptide is hydrophobic. At this time, as the absolute value increases, hydrophilicity or hydrophobicity increases. The expressions of those proteins were investigated. As a result, the soluble expression of Hepcidin I was observed only in those clones having MRRRRRRR sequence (pI 13.28, hydrophilicity value +1.97) (SEQ. ID. NO: 70) and MKKKKKKK sequence (pI 11.28, hydrophilicity value +1.97) (SEQ. ID. NO: 71) (see FIG. 5). These leader sequences retain the high pI value as a signal sequence (MRRRRRRR and MKKKKKKK) and the high pI value and high hydrophilicity as a secretional enhancer (RRRRRRR and KKKKKKK). This result is consistent with the description of Korean Patent Publication No. 10-2007-0009453 saying that the soluble expression of olive flounder Hepcidin I need the high pI value of the signal sequence and higher hydrophilicity value than that of amphipathic domain or transmembrane-like domain included in the sequence. However, in spite of similar sequences to those leader sequences (MRRRRRRR and MKKKKKKK), MKK(K)n(n=6)AK and M(R)n(n=8)AK sequences could hardly increase the soluble-expression of the adhesive protein Mefp1, compared with MAK, the control. Korean Patent Publication No. 10-2007-0009453 also describes that the soluble secretion of the adhesive protein Mefp1 could be slightly increased by substituting SmaI of pET-22b(+)(ompASP₁₋₈-SmaI-Xa-7×mefp1*) with the nucleotide corresponding to 6×Arg or 6×Lys, but the increase was not as significant as shown in the secretional enhancer sequence of olive flounder Hepcidin I (data not shown). Therefore, it is very difficult to judge whether or not these leader sequences of olive flounder Hepcidin I (MRRRRRRR and MKKKKKKK) are functioning as a signal sequence or a secretional enhancer or both (in the case that Met alone is functioning as a leader sequence, pI: 5.70).

To investigate the effect of the low pI value of the modified signal sequence on the soluble expression of olive flounder Hepcidin I, the present inventors prepared the protein in which signal sequence variants (MAH; pI7.65, MAA; pI5.60 or MEE; pI3.09), OmpASP₄₋₁₀-6×homologous amino acids and Xa recognition site (Xa) were linked to ofHepI in N-terminal of the protein and then constructed clones for the expression of the protein using the leader sequence having the controlled pI and hydrophobicity/hydrophilicity values (see Table 6). From the results of investigation of the soluble expressions of the clones, it was confirmed that the soluble protein was well expressed in pET-22b(+)[MAH(pI 7.65) -OmpASP₄₋₁₀-6×Arg-Xa-ofHep I**] and pET-22b(+)[MAA(pI 5.60) -OmpASP₄₋₁₀-6×Arg-Xa-ofHep I**], while the protein expression was weak in pET-22b(+)[MEE(pI 3.09)-OmpASP₄₋₁₀-6×Arg-Xa-ofHep I**]. However, the soluble expression was moderate in pET-22b(+)[MEE(pI 3.09) -OmpASP₄₋₁₀-6×Glu-Xa-ofHep I**] (see FIG. 6). The above results indicate that the soluble expression of olive flounder Hepcidin I is possibly induced not only in the case that the N-terminal of the protein is designed to have the signal sequence fragment (_{OmpASP}₁₋₁₀) with the high pI value (10.55) and 6×Arg and 6×Lys having the high pI value and high hydrophilicity as a secretion enhancer (Korean Patent Publication No. 10-2007-0009453) but also in the case that the N-terminal of the protein is designed to have the signal sequence fragment with the low pI value and 6×Glu having the low pI value but high hydrophilicity as a secretion enhancer.

By observing the soluble expression of olive flounder Hepcidin I, it was disclosed that the pI value of a signal sequence fragment and the pI value and hydrophilicity of a secretional enhancer sequence are closely related. That is, when the pI value of a signal sequence was 5.60, 7.65 and 10.55, a secretional enhancer comprising amino acids having the high pI value and high hydrophilicity was required, while when the pI value of the signal sequence fragment was as low as 3.09, not only a secretional enhancer comprising amino acids having the high pI value and high hydrophilicity but also another secretional enhancer comprising amino acids having the low pI value but high hydrophilicity could be used. So, it is pretty much likely that the pI value of a signal sequence fragment determines the characteristics of a secretional enhancer such as controlling the pI value and hydrophilicity, and thus the pI value of a signal sequence fragment is closely related to a secretional enhancer.

The above results are limited to the case when a secretional enhancer candidate sequence is directly linked to Met in N-terminal, the soluble expression is induced by Arg and Lys, the amino acids having the high pI value and high hydrophilicity. When the pI value of N-terminal of the signal sequence fragment containing hydrophobic region is controlled, not only the sequence comprising amino acids having the high pI value and high hydrophilicity but also the sequence comprising amino acids having the low pI value but high hydrophilicity, such as Glu, can be used as a secretional enhancer sequence, suggesting that the secretional enhancer sequence has a wide range of usability. So, the range of the hydrophilic secretional enhancer sequence can be expanded by lowering the hydrophilicity of N-terminal by linking a hydrophobic fragment to the N-terminal of the leader sequence with the controlled pI value.

This result also suggests that the pI value of the signal sequence fragment and the pI value of the modified signal sequence fragment have their own spectrum in olive flounder Hepcidin I. The margin of the pI value of the signal sequence fragment affects the functions of a secretional enhancer. So, when the pI value was controlled as low as 3.09 in the signal sequence, the soluble expression of Hepcidin I was induced by 6×Arg functioning as a secretional enhancer having the high pI value and high hydrophilicity and by 6×Glu functioning as another secretional enhancer having the low pI value but high hydrophilicity. At, the other pI values such as 5.60, 7.65, and 10.55, the soluble expression of the protein was induced only by 6×Arg functioning as a secretional enhancer having the high pI value and high hydrophilicity. However, when the pI value of the leader sequence was 3.09, 5.60, and 7.65, as shown in FIG. 2, the pI value was presumed to be involved in membrane permeation process, which was similar to the membrane permeation mechanism induced by the wide pI spectrum of the leader sequence of the adhesive protein Mefp1. However, when the pI value of the leader sequence was 10.55, as shown in FIG. 1, the soluble expression would be controlled by the OmpASP fragment specific pI value.

In conclusion, the pI value of the signal sequence fragment and the pI value of the leader sequence fragment played a critical role in the soluble expression of an adhesive protein Mefp1, but had nothing to do with electrical charge. The present inventors confirmed first the interrelationship between the soluble expression of an adhesive protein Mefp1 and the pI value of the leader sequence. Particularly, the present inventors found out the Lys specific membrane permeation mechanism (pI 9.90-11.41), the Arg specific membrane permeation mechanism (pI 11.52-13.35), the Asp/Glu specific membrane permeation mechanism (pI 2.73-3.25) and the non-specific membrane permeation mechanism (pI 3.25-9.90). However, when the secretional enhancer sequences poly Lys and poly Arg (Korean Patent Publication No. 10-2007-0009453) were linked to the leader sequence of an adhesive protein Mefp1, the expression of the protein was not much increased, suggesting that the binding between the leader sequence and the secretional enhancer does not affect the expression of such proteins which do not contain transmembrane-like domain. The present inventors also confirmed first that the optimum condition for the expression was when the leader sequence is linked to the factor Xa recognition site and when the distance between Lys-Lys in the signal sequence was properly controlled.

In olive flounder Hepcidin I having the transmembrane-like domain, the soluble expression was very weak or impossible only with the pI value of the signal sequence fragment and the pI value of the leader sequence. However, when a secretional enhancer candidate sequence was directly linked to Met, the soluble expression was induced by Arg and Lys having the high pI value and high hydrophilicity, and when the secretional enhancer sequence having the high pI value and hydrophilicity was linked to the signal sequence fragment with the controlled pI so as to have the wide pI spectrum, the soluble expression was induced. When the leader sequence having the low pI value was linked to a secretional enhancer comprising amino acids having the high pI value and hydrophilicity and a secretional enhancer comprising amino acids having the low pI value but high hydrophilicity, the expression was detected as well. This result supports the previous result that the expression of an adhesive protein Mefp1 can be induced in the wide pI spectrums of a signal sequence fragment and the leader sequence. But, secretional enhancer sequences generally need amino acids having the high hydrophilicity regardless of the pI value. Therefore, to induce the soluble expression, hydrophilicity has to be higher than that of the transmembrane-like domain in olive flounder Hepcidin I.

When the pI value of N-terminal of the signal sequence fragment containing a hydrophobic region was changed, the spectrum of the usable secretional enhancer sequence was broadened, compared with when the secretional enhancer candidate sequence was directly linked to Met. This result suggests that the hydrophobic region linked to the signal sequence lowers the hydrophilicity of N-terminal of the leader sequence (the sequence with the controlled pI value in N-terminal of the signal sequence), which makes the leader sequence be functioning freely as an anchor so that range of membrane permeation of the hydrophilic secretional enhancer sequence can be increased. And, it is presumed that there is a certain interaction between the pI value of N-terminal of the leader sequence and the secretional enhancer sequence.

Hereinafter, the preferable embodiments of the present invention are described in detail.

The present invention provides an expression vector for improving secretion efficiency of a foreign protein containing a gene construct which comprises (i) a promoter and (ii) a polynucleotide operably linked to the promoter encoding a polypeptide fragment containing a signal sequence and/or pI value of the N-region of the leader sequence of a foreign protein and/or N-region of the leader sequence or variants thereof in which the distance between amino acids affecting the pI value is controlled.

The promoter herein is preferably originated from virus, prokaryotes or eukaryotes. The virus originated promoter is exemplified by cytomegalovirus (CMV) promoter, polyoma virus promoter, fowl pox virus promoter, adenovirus promoter, bovine papilloma virus promoter, Avian sarcoma virus promoter, retrovirus promoter, hepatitis B virus promoter, herpes simplex virus thymidine kinase promoter and simian virus 40 (SV40) promoter, but not always limited thereto. The prokaryotes originated promoter is exemplified by T7 promoter, SP6 promoter, heat-shock protein 70 promoter, β-lactamase promoter, lactose promoter, alkaline phosphatase promoter, tryptophan promoter and tac promoter, but not always limited thereto. The eukaryotes originated promoter is preferably a yeast originated promoter, a plant originated promoter or an animal cell originated promoter. The yeast originated promoter is exemplified by 3-phosphoglycerate kinase promoter, enolase promoter, glyceraldehydes-3-phosphate dihydrogenase promoter, hexokinase promoter, pyruvate dicarboxylase promoter, phosphofructokinase promoter, glucose-6-phosphate isomerase promoter, 3-phosphoglycerate mutase promoter, pyruvate kinase promoter, triosphosphate isomerase promoter, phosphoglucose isomerase promoter, glucokinase promoter, alcohol dihydrogenase 2 promoter, isocytochrome C promoter, acidic phosphatase promoter, Saccharomyces cerevisiae GAL1 promoter, Saccharomyces cerevisiae GAL7 promoter, Saccharomyces cerevisiae GAL10 promoter and Pichia pastoris AOX1 promoter, but not always limited thereto. The animal cell originated promoter is exemplified by heat-shock protein promoter, proactin promoter and immuno globulin promoter, but not always limited thereto. In this invention, any promoter that is able to express a foreign gene in a host cell can be used.

The signal sequence herein is preferably originated from virus, prokaryotes or eukaryotes, which is exemplified by OmpA signal sequence, CT-B (cholera toxin subunit B) signal sequence, LTIIb-B (E. coli heat-labile enterotoxin B subunit) signal sequence, BAP (bacterial alkaline phosphatase) signal sequence (Izard and Kendall, Mol. Microbiol. 13:765-773, 1994), yeast carboxypeptidase Y signal sequence (Blachly-Dyson and Stevens, J. Cell. Biol. 104:1183-1191, 1987), Kluyveromyces lactis killer toxin gamma subunit signal sequence (Stark & Boyd, EMBO J 5(8):1995-2002, 1986), bovine growth hormone signal sequence (Lewin, B.(Ed), GENES V, p290. Oxford University Press, 1994), influenza neuraminidase signal-anchor (Lewin B(Ed), GENES V, p297. Oxford University Press, 1994), translocon-associated protein subunit alpha (TRAP-α) signal sequence (Prehn et al., Eur J Biochem 188(2):439-445, 1990) and twin-arginine translocation (Tat) signal sequence (Robinson, Biol Chem 381(2):89-93, 2000), but not always limited thereto.

The polypeptide fragment containing N-region with the controlled pI value is preferably the polypeptide composed of 1-6 amino acids with the controlled pI value of 9.90-11.41 or the polypeptide composed of 1-12 amino acids with the controlled pI value of 3.09-9.90, but not always limited thereto. An amino acid of the peptide can be substituted with another amino acid or the sequence and length of the amino acid of the peptide can be modified by considering the pI value of the N-region. In a preferred embodiment of the present invention, the pI value of the polypeptide fragment containing N-region of the signal sequence can be screened for the soluble expression of a foreign protein (see FIG. 1 and Table 1). In the meantime, the distance between amino acids that can affect the pI value in the leader sequence can be regulated for the optimum soluble expression of a foreign protein (see FIGS. 1-4 and Tables 1-4).

The pI value of the polypeptide fragment can be controlled by inserting additional amino acids that can change the pI value in between amino acids of N-region. Particularly, the pI value of the fragment can be increased by inserting an additional basic amino acid such as Lys, Arg and His, etc. On the contrary the pI value can be reduced by adding an acidic amino acid such as Asp and Glu. To regulate the distance between amino acids, which is an important factor affecting the pI value, a non-polar neutral amino acid selected from the group consisting of Gln, Ala, Val, Leu, Ile, Phe, Trp, Met, Cys and Pro, or a polar neutral amino acid selected from the group consisting of Ser, Thr, Tyr, Asn and Gln can be additionally added. The method for substituting amino acids is well known to those in the art (Sambrook et al., 1989. “Molecular Cloning: A Laboratory Manual”, 2nd ed. Cold Spring Harbor Laboratory Press, Cold Spring Harbor, N.Y.).

In a preferred embodiment of the present invention, a polynucleotide encoding the protease recognition site was operably linked to a polynucleotide encoding the polypeptide fragment having N-region with the controlled pI value (see Table 3). The protease recognition site herein can be one of Xa factor recognition site, enterokinase recognition site, genenase I recognition site and furin recognition site or can be composed of two or more recognition sites arranged in order. If the protease recognition site is the factor Xa recognition site, it is preferably composed of Ile-Glu-Gly-Arg. It is also preferred to insert a non-polar neutral amino acid selected from the group consisting of Gln, Ala, Val, Leu, Ile, Phe, Trp, Met, Cys and Pro or a polar neutral amino acid selected from the group consisting of Ser, Thr, Tyr, Asn and Gln in between the polynucleotide encoding the polypeptide fragment containing N-region with the controlled pI value and the nucleotide encoding the protease recognition site, to regulate the distance between amino acids as 0-2. In a preferred embodiment of the present invention, the optimum distance was generated by linking the protease recognition site to the polynucleotide encoding the polypeptide fragment having N-region with the controlled pI value (see Table 3 and FIG. 3).

In another preferred embodiment of the present invention, the expression vector of the invention additionally includes a protease recognition site for the insertion of a gene encoding a foreign protein. The protease recognition site herein is linked behind the polynucleotide encoding the polypeptide fragment containing N-region of the signal sequence with the controlled pI value. In the vector containing the polynucleotide encoding a secretional enhancer, the site is linked behind the polynucleotide. In the vector containing the polynucleotide encoding the protease recognition site, the protease recognition site might or might not be added. It might be unfavorable to clone a gene encoding a foreign protein by using a protease recognition site to produce a protein in native form.

A gene encoding a foreign protein can be additionally included in the said vector. The foreign protein is not limited and any protein preferred by those in the art can be accepted. And, a protein selected from the group consisting of antigen, antibody, cell receptor, enzyme, structural protein, serum, and cellular protein can be expressed as a recombinant fusion protein. The foreign protein herein is preferably the protein not containing one or more of transmembrane domain, transmembrane-like domain and amphipathic domain, which is preferably Mefp1 polymer, but not always limited thereto.

The present invention also provides an expression vector for improving secretion efficiency of a foreign protein containing a gene construct which comprises (i) a promoter; (ii) a polynucleotide operably linked to the promoter encoding a polypeptide fragment containing a segment of signal sequence and/or N-region of the leader sequence of a foreign protein or variants thereof in which the pI value is controlled ; and (iii) a polynucleotide operably linked to the polypeptide fragment or variants thereof encoding a secretional enhancer comprising a hydrophilicity enhancing sequence with the controlled pI value.

The foreign protein above is preferably the protein that contains transmembrane domain, transmembrane-like domain or amphipathic domain. It is suggested that the region having+charge of the protein containing transmembrane domain, transmembrane-like domain or amphipathic domain is adhered onto lipid bilayer of a membrane and this structure plays an anchor-like role to inhibit extracellular secretion. The expression vector of the present invention favors the extracellular secretion of such proteins having a difficulty in extracellular secretion. So, the vector of the present invention is suitable for the soluble production of a protein having transmembrane domain, transmembrane-like domain or amphipathic domain. When the modified signal sequence has directional signal and higher hydrophilicity than the transmembrane domain of a foreign target protein has, a nascent polypeptide is secreted out of periplasm. This seems to be because the directional signal of a signal sequence and hydrophilicity is higher than the tendency to adhere lipid bilayer, so that the secretion of the domain is accelerated. The foreign protein containing transmembrane domain, transmembrane-like domain or amphipathic domain is preferably olive flounder Hepcidin I, but not always limited thereto.

If a protein is identified as the one having transmembrane domain, transmembrane-like domain or amphipathic domain by hydrophobic (hydropathic) profile analysis detecting transmembrane-like domain or the sequence composed of a series of multiple hydrophilic amino acids behind the sequence composed of a series of multiple hydrophobic amino acids, this protein can be applied in the expression system of the present invention. For the decision, such computer soft wares as DNASIS™ (Hitachi, Japan), DOMpro (Cheng et al., Knowledge Discovery and Data Mining 13(1):1-20, 2006; //www.ics.uci.edu/˜baldig/dompro.html), TMpred (//www.ch.embnet.org/software/TMPRED_form.html), HMMTOP (//www.enzim.hu/hmmtop/html/submit.html), TBBpred (//www.imtech.res.in/raghava/tbbpred/) and DAS-TMfilter (//www.enzim.hu/DAS/DAS.html) can be used.

The pI value of the secretional enhancer of (iii) of the present invention is preferably changed by the pI value of the polypeptide fragment of (ii). Particularly, when the pI value of the polypeptide fragment is controlled to 5-11, the pI value of the secretional enhancer is preferably controlled to 11-14. When the pI value of the polypeptide fragment is controlled to 2-5, the pI value of the secretional enhancer is preferably controlled to 2-14. The polypeptide fragment can additionally include a basic amino acid selected from the group consisting of Lys, Arg and His or one of the inside acidic amino acids of the fragment can be substituted with a basic amino acid to increase the pI value. Or an acidic amino acid such as Asp or Glu can be additionally inserted into the fragment or one of the inside basic amino acids can be substituted with an acidic amino acid to reduce the pI value. In a preferred embodiment of the present invention, from the examination of the soluble expression of olive flounder Hepcidin I, it was confirmed that the pI value of the signal sequence and the pI value of the secretional enhancer sequence and hydrophilicity are closely related one another. When the pI value of the signal sequence fragment is 5.60, 7.65 or 10.55, a secretional enhancer comprising amino acids having the high pI value and high hydrophilicity is required. In the meantime, when the pI value of the signal sequence fragment is as low as 3.09, a secretional enhancer comprising amino acids having the high pI value and high hydrophilicity and/or another secretional enhancer comprising amino acids having the low pI value but high hydrophilicity can be used (see Table 5, Table 6, FIG. 5 and FIG. 6).

The polynucleotide encoding a secretional enhancer can be operably linked to the polynucleotide encoding a polypeptide fragment having N-region of the vector of the invention with the controlled pI value. The secretional enhancer herein is composed of a hydrophilicity enhancing sequence with the controlled pI value, by which the hydrophilicity of a signal sequence is increased to induce secretion of a foreign protein out of periplasm. The secretional enhancer herein is a hydrophilic peptide composed of at least 60%, preferably at least 65%, and more preferably at least 70% of hydrophilic amino acids, and the length thereof is not limited, but preferably 2-50 amino acids, and more preferably 4-25 amino acids, and most preferably 6-20 amino acids long. One of the most preferable examples of the enhancer is the polypeptide having the repeats of 6 hydrophilic amino acids. Herein, the hydrophilic amino acid is not limited but preferably Asn, Gln, Ser, Lys, Arg, Asp or Glu, and more preferably Lys, Arg, Glu or Asp. The pI value of the secretional enhancer can be screened for the soluble expression of a foreign protein. That is, the sequence and the lengths of amino acids of the signal sequence and the leader sequence of the said polypeptide can be regulated based on the control of the pI value of the secretional enhancer to a specific range favoring the soluble expression of a foreign protein (see Table 5, Table 6, FIG. 5 and FIG. 6).

In the expression vector of the present invention, the polynucleotide encoding a secretional enhancer is inserted in between the polynucleotide encoding the polypeptide fragment having N-region with the controlled pI value and the polynucleotide encoding a protease recognition site (see Table 6). And this insertion is preferably performed by the protease recognition site digested with a protease generating blunt ends such as Smal. The protease recognition site is selected from the group consisting of factor Xa recognition site, enterokinase recognition site, genenase I recognition site and furin recognition site. The protease recognition site can be used alone or used as being fused.

In a preferred embodiment of the present invention, the expression vector of the present invention contains a gene construct linked to the polynucleotide encoding a secretional enhancer, a protease recognition site for the insertion of a foreign gene, and the polynucleotide encoding the foreign protein operably linked to the gene construct. The foreign gene can be cloned into the protease recognition site. The expression vector of the present invention can additionally include the polynucleotide encoding a protease recognition site. At this time, this polynucleotide is linked to the above said polynucleotide in frame so as to produce a native form of the inserted foreign protein after secretion and cleaving with the protease.

The present invention also provides a transformant produced by transforming a host cell with the said expression vector.

The host cell herein is preferably a prokaryotic cell or an eukaryotic cell, but not always limited thereto. And, the prokaryotic cell herein is preferably selected from the group consisting of virus, E. coli and Bacillus, but not always limited thereto. The eukaryotic cell herein is preferably a mammalian cell, an insect cell, yeast or a plant cell, but not always limited thereto.

The present invention also provides a method for improving secretion efficiency of a recombinant protein using the said transformant.

Particularly, the present invention provides a method for improving secretion efficiency of a foreign protein comprising the following steps:

1) designing a leader sequence having a signal sequence and/or N-region of the leader sequence of a foreign protein with the controlled pI value of 9.90-11.28;

2) regulating the distance between amino acids which affect the pI value in the leader sequence;

3) constructing a gene construct composed of a polynucleotide encoding a fusion protein containing the leader sequence of step 1), the controlled distance region of step 2), a protease recognition site and the foreign protein in that order;

4) constructing a recombinant expression vector by inserting operably the gene construct of step 3) into a general expression vector;

5) generating a transformant by transforming a host cell with the recombinant expression vector of step 4); and,

6) selecting a transformant from the culture of the transformant of step 5) exhibiting the highest soluble expression of the target protein.

Also, the present invention provides a method for improving secretion efficiency of a foreign protein comprising the following steps:

1) designing a leader sequence having a signal sequence and/or N-region of the leader sequence of a foreign protein with the controlled pI value of 3.09-9.90;

2) constructing a gene construct composed of a polynucleotide encoding a fusion protein containing the leader sequence of step 1), a protease recognition site and the foreign protein in that order;

3) constructing a recombinant expression vector by inserting the gene construct of step 2) operably into a general expression vector;

4) generating a transformant by transforming a host cell with the recombinant expression vector of step 3); and,

5) selecting a transformant from the culture of the transformant of step 4) exhibiting the highest soluble expression of the target protein.

In a preferred embodiment of the present invention, the pI value of the leader sequence was controlled for the soluble expression of the adhesive protein Mefp1. As a result, when the pI value was controlled to 9.90-11.41 (see Table 1 and FIG. 1) or when the pI value was controlled to 3.09-9.90, the soluble expression of the protein was most significant (see Table 2 and FIG. 2). Also, the pI value of the leader sequence was controlled to 10.55 and 10.82 and the distance between Lys-Lys affecting the pI value was regulated by adding an amino acid not affecting the pI value. As a result, the present inventors determined the optimum distance for the expression (see Table 4 and FIG. 4).

The amino acid affecting the pI value in step 2) is preferably Lys.

The foreign protein herein is not limited and can be any protein that can be accepted by those in the art, which can be expressed as a recombinant fusion protein using a protein selected from the group consisting of antigen, antibody, cell receptor, enzyme, structural protein, serum, and cellular protein. The foreign protein is preferably the protein that does not contain transmembrane domain, transmembrane-like domain or amphipathic domain, which is preferably Mefp1 polymer, but not always limited thereto.

The present invention also provides a method for improving secretion efficiency of a foreign protein comprising the following steps:

1) designing a leader sequence having a signal sequence and/or N-region of the leader sequence of a foreign protein with the controlled pI value of 2-5;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3); and,

5) selecting a transformant from the culture of the transformant of step 4) exhibiting the highest soluble expression of the target protein.

The present invention also provides a method for producing a recombinant fusion foreign protein comprising the following steps:

1) designing a leader sequence having a signal sequence and/or N-region of the leader sequence of a foreign protein with the controlled pI value of 9.90-11.28;

2) regulating the distance between amino acids which affect the pI value in the leader sequence;

3) constructing a gene construct composed of a polynucleotide encoding a fusion protein containing the leader sequence of step 1), the controlled distance region of step 2), a protease recognition site and the foreign protein in that order;

4) constructing a recombinant expression vector by inserting operably the gene construct of step 3) into a general expression vector;

5) generating a transformant by transforming a host cell with the recombinant expression vector of step 4);

6) culturing the transformant of step 5); and,

7) separating the recombinant fusion foreign protein from the culture solution of step 6).

The foreign protein herein is preferably the protein that contains transmembrane domain, transmembrane-like domain or amphipathic domain, but not always limited thereto.

The pI value of the secretional enhancer of the present invention is preferably changed by the pI value of the polypeptide fragment. Particularly, when the pI value of the polypeptide fragment is controlled to 5-11, the pI value of the secretional enhancer is preferably controlled to 11-14. When the pI value of the polypeptide fragment is controlled to 2-5, the pI value of the secretional enhancer is preferably controlled to 2-14. The polypeptide fragment can additionally include a basic amino acid selected from the group consisting of Lys, Arg and His or one of the inside acidic amino acids of the fragment can be substituted with a basic amino acid to increase the pI value. Or an acidic amino acid such as Asp or Glu can be additionally inserted into the fragment or one of the inside basic amino acids can be substituted with an acidic amino acid to reduce the pI value. In a preferred embodiment of the present invention, from the examination of the soluble expression of olive flounder Hepcidin I, it was confirmed that the pI value of the signal sequence and the pI value of the secretional enhancer sequence and hydrophilicity are closely related one another. When the pI value of the signal sequence fragment is 5.60, 7.65 or 10.55, a secretional enhancer comprising amino acids having the high pI value and high hydrophilicity is required. In the meantime, when the pI value of the signal sequence fragment is as low as 3.09, a secretional enhancer comprising amino acids having the high pI value and high hydrophilicity and/or another secretional enhancer comprising amino acids having the low pI value but high hydrophilicity can be used (see Table 5, Table 6, FIG. 5 and FIG. 6).

The present invention also provides a method for producing a recombinant fusion foreign protein comprising the following steps:

1) designing a leader sequence having a signal sequence and/or N-region of the leader sequence of a foreign protein with the controlled pI value of 9.90-11.28;

2) regulating the distance between amino acids which affect the pI value in the leader sequence;

3) constructing a gene construct composed of a polynucleotide encoding a fusion protein containing the leader sequence of step 1), the controlled distance region of step 2), a protease recognition site and the foreign protein in that order;

4) constructing a recombinant expression vector by inserting operably the gene construct of step 3) into a general expression vector;

5) generating a transformant by transfoming a host cell with the recombinant expression vector of step 4);

6) culturing the transformant of step 5); and,

7) separating the recombinant fusion foreign protein from the culture solution of step 6).

The present invention also provides a method for producing a recombinant fusion foreign protein comprising the following steps:

1) designing a leader sequence having a signal sequence and/or N-region of the leader sequence of a foreign protein with the controlled pI value of 3.09-9.90;

2) constructing a gene construct composed of a polynucleotide encoding a fusion protein containing the leader sequence of step 1), a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4); and,

6) separating the recombinant fusion foreign protein from the culture solution of step 5).

The recombinant fusion foreign protein herein can be produced by expressing the protein in a transformant transformed with the said expression vector and recovering the protein therefrom. The method for recovering the protein can be selected among the conventional methods known to those in the art.

The foreign protein is not limited and any protein preferred by those in the art can be accepted. And, protein selected from the group consisting of antigen, antibody, cell receptor, enzyme, structural protein, serum, and cellular protein can be expressed as a recombinant fusion protein. The foreign protein herein is preferably the protein not containing transmembrane domain, transmembrane-like domain or amphipathic domain, which is preferably Mefp1 polymer, but not always limited thereto.

The present invention also provides a method for producing a recombinant fusion foreign protein comprising the following steps:

1) designing a leader sequence having a signal sequence and/or N-region of the leader sequence of foreign protein with the controlled pI value of 5-11;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4); and,

6) separating the recombinant fusion foreign protein from the culture solution of step 5).

The present invention also provides a method for producing a recombinant fusion foreign protein comprising the following steps:

1) designing a leader sequence having a signal sequence and/or N-region of the leader sequence of a foreign protein with the controlled pI value of 2-5;

2) constructing a gene construct composed of polynucleotide encoding a fusion protein containing the leader sequence of step 1), a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4); and,

6) separating the recombinant fusion foreign protein from the culture solution of step 5).

The foreign protein herein is preferably the protein that contains transmembrane domain, transmembrane-like domain or amphipathic domain, but not always limited thereto.

If a therapeutic protein targeting the brain, for example beta-amyloid specific scFv (single chain variable fragment), is used as a foreign protein, the fusion protein generated from fusion of the modified signal sequence and the foreign protein by the method of the present invention can pass through blood-brain barrier to work directly on the brain, unlike general proteins. So, the method of the present invention can be a dramatic momentum in the advancement of a drug delivery system for treating brain disease. In addition to the advantage of passing through blood-brain barrier, the fusion foreign protein prepared by the method of the present invention can be delivered wherever in the body because it can pass through the stomach wall before being decomposed in the stomach and can pass through the skin and be smeared into the body when applied or patched on the skin. Therefore, the method of the present invention overcomes the limitations of the conventional protein preparations in administration method (intravenous injection, intramuscular injection, hypodermic injection or nasal administration), so that it facilitates simpler administration methods such as oral administration and transdermal administration.

The present invention provides a recombinant fusion foreign protein produced by the said method.

The foreign protein herein is not limited but the therapeutic protein targeting the brain is preferred. The recombinant fusion foreign protein produced by the method of the present invention harbors transmembrane domain that can pass through blood-brain barrier by containing a modified signal sequence. Further, the present invention also provides a pharmaceutical composition containing the recombinant fusion foreign protein of a modified signal sequence and the foreign protein and a pharmaceutically acceptable carrier. The pharmaceutical composition is preferably used for the treatment of brain disease, but not always limited thereto.

The present invention also provides a pharmaceutical composition containing the recombinant fusion foreign protein and a pharmaceutically acceptable carrier.

The pharmaceutical composition is expected to increase the efficiency in delivery of the conventional therapeutic protein for brain disease such as stroke and senile dementia (Alzheimer's disease).

The pharmaceutical composition of the present invention can be administered by any conventional pathway that can deliver the drug into a target area, particularly by local, oral, parenteral, intranasal, intravenous, intramuscular, hypodermic, ophthalmic or transdermal administration. This composition can be formulated as solutions, suspensions, tablets, pills, capsules and sustained-release preparations, and injectable solutions are preferred. To prepare injectable solutions, sterilized isotonic solution or saline can be added, and the injectable solution can be administered by hypodermic injection, intramuscular injection and intravenous injection. The effective dosage of the composition can be determined by those in the art by considering the severity and the type of a disease, age, gender, administration method, target cells, expression levels, etc.

The pharmaceutical composition of the present invention can additionally include a pharmaceutically acceptable carrier, for example, an excipient, a disintegrating agent, a sweetening agent, a lubricant and a flavor. The disintegrating agent is exemplified by sodium starch glycolate, crospovidone, croscarmellose sodium, alginic acid, calcium carboxymethyl cellulose, sodium carboxymethyl cellulose, chitosan, guar gum, low-substituted hydroxypropyl cellulose, magnesium aluminum silicate, polacrilin potassium, etc. The pharmaceutical composition of the present invention can additionally include a pharmaceutically acceptable additive, which is exemplified by starch, gelatinized starch, microcrystalline cellulose, lactose, povidone, colloidal silicon dioxide, calcium hydrogen phosphate, lactose, mannitol, taffy, Arabia rubber, pregelatinized starch, corn starch, cellulose powder, hydroxypropyl cellulose, Opadry, carunauba wax, synthetic aluminum silicate, stearic acid, magnesium stearate, aluminum stearate, calcium stearate, white sugar, dextrose, sorbitol, talc, etc. The pharmaceutically acceptable additive herein is preferably added by 0.1-90 weight part to the pharmaceutical composition.

Solid formulations for oral administration are powders, granules, tablets, capsules, soft capsules and pills. Liquid formulations for oral administrations are suspensions, solutions, emulsions, syrups and aerosols, and the above-mentioned formulations can contain various excipients such as wetting agents, sweeteners, aromatics and preservatives in addition to generally used simple diluents such as water and liquid paraffin. For formulations for parenteral administration, powders, granules, tablets, capsules, sterilized suspensions, liquids, water-insoluble excipients, suspensions, emulsions, syrups, suppositories, external use such as aerosols and sterilized injections can be prepared by the conventional, and preferably skin external pharmaceutical compositions such as creams, gels, patches, sprays, ointments, plasters, lotions, liniments, pastes or cataplasms can be prepared, but not always limited thereto. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The effective dosage of the pharmaceutical composition of the present invention can be determined according to absorptiveness of the active ingredient, inactivation rate, excretion rate, age, gender, health condition and severity of a disease by those in the art. In the case of oral administration, the pharmaceutical composition can be administered by 0.0001-100 mg/kg per day for an adult, and more preferably by 0.001-100 mg/kg per day. The administration frequency is once a day or a few times a day. The said dosage cannot limit the scope of the present invention by any means.

The present invention also provides a method for producing a foreign protein in native form.

Particularly, the present invention provides a method for producing a foreign protein in native form comprising the following steps:

1) designing a leader sequence having a signal sequence and/or N-region of the leader sequence of a foreign protein with the controlled pI value of 9.90-11.28;

2) regulating the distance between amino acids which affect the pI value in the leader sequence;

3) constructing a gene construct composed of a polynucleotide encoding a fusion protein containing the leader sequence of step 1), the controlled distance region of step 2), a protease recognition site and the foreign protein in that order;

4) constructing a recombinant expression vector by inserting operably the gene construct of step 3) into a general expression vector;

5) generating a transformant by transforming a host cell with the recombinant expression vector of step 4);

6) culturing the transformant of step 5);

7) separating the recombinant fusion foreign protein from the culture solution of step 6); and,

8) separating the foreign protein in native form after cleaving the fusion foreign protein of step 7) with a protease that could cleave the protease recognition site.

The foreign protein herein is preferably the protein that does not contain one or more of transmembrane domain, transmembrane-like domain or amphipathic domain, and the amino acid affecting the pI value of step 2) can be Lys.

Particularly, the present invention also provides a method for producing a foreign protein in native form comprising the following steps:

1) designing a leader sequence having a signal sequence and/or N-region of the leader sequence of a foreign protein with the controlled pI value of 3.09-9.90;

2) constructing a gene construct composed of a polynucleotide encoding a fusion protein containing the leader sequence of step 1), a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5); and,

7) separating the foreign protein in native form after cleaving the fusion foreign protein of step 6) with a protease that could cleave the protease recognition site.

The foreign protein herein is characteristically the protein that does not contain one or more of transmembrane domain, transmembrane-like domain or amphipathic domain.

The present invention also provides a method for producing a foreign protein in native form comprising the following steps:

1) designing a leader sequence having a signal sequence and/or N-region of the leader sequence of a foreign protein with the controlled pI value of 5-11;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5); and,

7) separating the foreign protein in native form after cleaving the fusion foreign protein of step 6) with a protease that could cleave the protease recognition site.

The foreign protein herein is characteristically the protein that contains one or more transmembrane domain, transmembrane-like domain or amphipathic domain, and the pI value of the hydrophilic secretional enhancer of step 2) is controlled to 11-14.

The present invention also provides a method for producing a foreign protein in native form comprising the following steps:

1) designing a leader sequence having a signal sequence and/or N-region of the leader sequence of a foreign protein with the controlled pI value of 2-5;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5); and,

7) separating the foreign protein in native form after cleaving the fusion foreign protein of step 6) with a protease that could cleave the protease recognition site.

The foreign protein herein is characteristically the protein that contains one or more transmembrane domain, transmembrane-like domain or amphipathic domain, and the pI value of the hydrophilic secretional enhancer of step 2) is controlled to 2-14.

The present invention also provides a method for producing an intracellular carrier for the delivery of a target material into the cell.

Particularly, the present invention provides a method for producing an intracellular carrier for the delivery of a target material comprising the following steps:

1) designing a leader sequence having a signal sequence and/or N-region of the leader sequence of a foreign protein with the controlled pI value of 9.90-11.28;

2) regulating the distance between amino acids which affect the pI value in the leader sequence;

3) constructing a gene construct composed of a polynucleotide encoding a fusion protein containing the leader sequence of step 1), the controlled distance region of step 2), a protease recognition site and the foreign protein in that order;

4) constructing a recombinant expression vector by inserting operably the gene construct of step 3) into a general expression vector;

5) generating a transformant by transforming a host cell with the recombinant expression vector of step 4);

6) culturing the transformant of step 5);

7) separating the fusion foreign protein from the culture solution of step 6);

8) separating the peptide containing the leader sequence, the hydrophilic secretional enhancer and the protease recognition site but not the foreign protein in native form after cleaving the fusion foreign protein of step 7) with a protease that could cleave the protease recognition site; and,

9) combining the peptide containing the leader sequence, the hydrophilic secretional enhancer and the protease recognition site of step 8) with a target material which is supposed to be delivered into the cell.

The foreign protein herein is preferably the protein that does not contain one or more of transmembrane domain, transmembrane-like domain and amphipathic domain, and the amino acid affecting the pI value of step 2) can be Lys.

The material supposed to be delivered into the cell herein is preferably selected from the group consisting of natural compounds, synthetic compounds, RNA, DNA, polypeptides, antisense peptide nucleic acids, enzymes, proteins, ligands, antibodies, antigens, metabolites of bacteria or fungi and bioactive molecules, but not always limited thereto.

The present invention also provides a method for producing an intracellular carrier for the delivery of a target material comprising the following steps:

1) designing a leader sequence having a signal sequence and/or N-region of the leader sequence of a foreign protein with the controlled pI value of 3.09-9.90;

2) constructing a gene construct composed of a polynucleotide encoding a fusion protein containing the leader sequence of step 1), a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5);

7) separating the peptide containing the leader sequence, the hydrophilic secretional enhancer and the protease recognition site but not the foreign protein in native form after cleaving the fusion foreign protein of step 6) with a protease that could cleave the protease recognition site; and

8) combining the peptide containing the leader sequence, the hydrophilic secretional enhancer and the protease recognition site of step 7) with a target material which is supposed to be delivered into the cell.

The foreign protein herein is preferably the protein that does not contain one or more of transmembrane domain, transmembrane-like domain and amphipathic domain.

The material supposed to be delivered into the cell herein is preferably selected from the group consisting of natural compounds, synthetic compounds, RNA, DNA, polypeptides, antisense peptide nucleic acids, enzymes, proteins, ligands, antibodies, antigens, metabolites of bacteria or fungi and bioactive molecules, but not always limited thereto.

The present invention also provides a method for producing an intracellular carrier for the delivery of a target material comprising the following steps:

1) designing a leader sequence having a signal sequence and/or N-region of the leader sequence of a foreign protein with the controlled pI value of 5-11;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5);

7) separating the peptide containing the leader sequence, the hydrophilic secretional enhancer and the protease recognition site but not the foreign protein in native form after cleaving the fusion foreign protein of step 6) with a protease that could cleave the protease recognition site; and

8) combining the peptide containing the leader sequence, the hydrophilic secretional enhancer and the protease recognition site of step 7) with a target material which is supposed to be delivered into the cell.

The foreign protein herein is characteristically the protein that contains one or more of transmembrane domain, transmembrane-like domain and amphipathic domain, and the pI value of the hydrophilic secretional enhancer of step 2) is controlled to 11-14.

The material supposed to be delivered into the cell herein is preferably selected from the group consisting of natural compounds, synthetic compounds, RNA, DNA, polypeptides, antisense peptide nucleic acids, enzymes, proteins, ligands, antibodies, antigens, metabolites of bacteria or fungi and bioactive molecules, but not always limited thereto.

The present invention also provides a method for producing an intracellular carrier for the delivery of a target material comprising the following steps:

1) designing a leader sequence having a signal sequence and/or N-region of the leader sequence of a foreign protein with the controlled pI value of 2-5;

2) constructing a gene construct composed of a polynucleotide encoding a fusion protein containing the leader sequence of step 1), a hydrophilic secretional enhancer, a protease recognition site and the foreign protein in that order;

3) constructing a recombinant expression vector by inserting the gene construct of step 2) operably into a general expression vector;

4) generating a transformant by transforming a host cell with the recombinant expression vector of step 3);

5) culturing the transformant of step 4);

6) separating the fusion foreign protein from the culture solution of step 5);

7) separating the peptide containing the leader sequence, the hydrophilic secretional enhancer and the protease recognition site but not the foreign protein in native form after cleaving the fusion foreign protein of step 6) with a protease that could cleave the protease recognition site; and

8) combining the peptide containing the leader sequence, the hydrophilic secretional enhancer and the protease recognition site of step 7) with a target material which is supposed to be delivered into the cell.

The foreign protein herein is characteristically the protein that contains one or more of transmembrane domain, transmembrane-like domain and amphipathic domain, and the pI value of the hydrophilic secretional enhancer of step 2) is controlled to 2-14.

The material supposed to be delivered into the cell herein is preferably selected from the group consisting of natural compounds, synthetic compounds, RNA, DNA, polypeptides, antisense peptide nucleic acids, enzymes, proteins, ligands, antibodies, antigens, metabolites of bacteria or fungi and bioactive molecules, but not always limited thereto.

### Mode for Invention

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Cloning of Adhesive Protein Gene DNA Multimer Cassette

The present inventors constructed synthetic mefp1 DNA based on Mepf1 having the same sequence with that described in Korean Patent Publication No. 10-2007-0009453 and being represented by SEQ. ID. NO: 95 (Ala Lys Pro Ser Tyr Pro Pro Thr Tyr Lys), with the forward primer represented by SEQ. ID. NO: 96 (5′-TAC AAA GCT AAG CCG TCT TAT CCG CCA ACC-3′) which was the same as the one used in Korean Patent Publication No. 10-2007-0009453 and the reverse primer represented by SEQ. ID. NO: 97 (5′-TTT GTA GGT TGG CGG ATA AGA CGG CTT AGC-3′) which was the same as the one used in Korean Patent Publication No. 10-2007-0009453. Left adaptor (referred as “La” hereinafter) synthetic DNA was synthesized by using the forward primer represented by SEQ. ID. NO: 98 (5′-GAT CCG AAT TCC CCG GG-3′) harboring BamHI/EcoRI/SmaI sites which was the same as the one used in Korean Patent Publication No. 10-2007-0009453 and the reverse primer represented by SEQ. ID. NO: 99 (5′-TTT GTA CCC GGG GAA TTC G-3′) which was the same as the one used in Korean Patent Publication No. 10-2007-0009453. In the mean time, right adaptor (referred as “Ra” hereinafter) synthetic DNA was synthesized by using the forward primer represented by SEQ. ID. NO: 100 (5′-TAC AAA CGT AAG CTT GTC GAC C-3′) harboring Arg/HindIII/SalI/XhoI sites which was the same as the one used in Korean Patent Publication No. 10-2007-0009453 and the reverse primer represented by SEQ. ID. NO: 101 (5′-TCG AGG TCG ACA AGC TTA CG-3′) which was the same as the one used in Korean Patent Publication No. 10-2007-0009453. The mefp1 DNA multimer was prepared by the method described in Korean Patent No. 379,025, which was then cloned into pBluescriptIISK(+) vector (Stratagene, USA). The mefp1 DNA multimer which was repeated 7 times was screened and named ‘pBluescriptIISK(+)La-7×mefp1-Ra’.

### Example 2

### Expression of an Adhesive Protein in the N-Terminal Variant Clone

The present inventors performed PCR using pBluescriptIISK(+)La-7×mefp1-Ra as a template to introduce the OmpA signal peptide (OmpASP) fragment for the soluble expression according to the controlled pI value of the N-terminal of Mefp1. As a result, expression vectors having N-terminal were constructed by linking pET-22b(+) vector with the OmpASP fragment or its variants having the different pI values, the leader sequence of Mefp1 and the mefp1 cassette prepared in Example 1 (Table 1-Table 4).

E. coli BL21 (DE3) was transformed with the expression vectors containing N-terminal constructed as shown in Table 1-Table 4 according to the conventional method, followed by culture in LB medium (tryptone 10 g, yeast extract 5 g, NaCl 10 g/l) supplemented with 50 μg/ml of ampicillin at 30° C. for 16 hours. The culture solution was diluted 200 times with the LB medium. 1 mM of IPTG was added to the diluted culture solution, followed by culture until OD₆₀₀ reached 0.3. Culture continued for three more hours. 1 ml of the culture solution proceeded to centrifugation at 4° C., 4,000×g for 30 minutes and the pellet was resuspended in 100-200 μl of PBS. The suspension was homogenized to separate a protein by using a sonicator at 15× 2-s cycle pulses (at 30% power output). Centrifugation was performed at 4° C., 16,000 rpm for 30 minutes to eliminate cell debris, resulting in the separation of an insoluble fraction. The protein of a soluble fraction was quantified by Bradford method (Bradford, Anal Biochem 72:248-254, 1976), followed by SDS-PAGE by using 15% SDS-PAGE gel according to the method of Laemmli et al (Laemmli, Nature 227:680-685, 1970). Coomassie Brilliant Blue (Sigma, USA) staining was performed. The SDS-PAGE gel was transferred onto a nitrocellulose membrane (Roche, USA). After dipping in 5% skim milk (skimmed milk; Difco, USA), the membrane was soaked in 0.4 μg/ml of anti-His6 monoclonal antibody solution (Santa Cruz Biotechnology, USA) at 37° C. for 2 hours. DAB (3,3′-diaminobenzidine tetrahydrochloride, Sigma, USA) staining was performed using horseradish peroxidase conjugated rabbit anti-mouse IgG (Santa Cruz Biotechnology, USA) as a secondary antibody. The concentration of the adhesive protein Mefp1 band obtained thereby was measured by densitometer analysis using Quantity One program (Bio Rad, USA).

### Example 3

### Effect of a Short Signal Sequence Fragment having the Increased pI Value and its Variants on the Expression of an Adhesive Protein

5′-end of the nucleotide sequence 7×mefp1 encoding the adhesive foreign protein Mefp1 was fused with coding sequences of OmpASP₁(Met), OmpASP₁₋₂(Met-Lys) and OmpASP₁₋₃(Met-Lys-Lys), the fragments of OmpA signal peptide (OmpASP, Korean Patent Publication No. 10-2007-0009453, SEQ. ID. NO: 46 or Movva et al., J Biol Chem 255, 27-29, 1980) inducing the protein secretion, resulting in the construction of the clones pET-22b(+)(OmpASP₁-7×mefp1*), pET-22b(+)(OmpASP₁₋₂-7×mefp1*) and pET-22b(+)(OmpASP₁₋₃-7×mefp1*) (Table 1).

E. coli BL21 (DE3) was transformed with the clone vectors constructed above by the same manner as described in Example 2, and the protein expression was quantified. As a result, the change of one amino acid (Lysine; Lys; K; pI=9.72) made a significant difference in the soluble expression of Met-7×Mefp1* (SEQ. ID. NO: 15) and Met-Lys-7×mefp1* (SEQ. ID. NO: 16) from the above two clones (FIG. 1a, line 1 and line 2, Table 1). The above result indicates that the amino acid Lys affects significantly the expression of the adhesive protein Mefp1 at the N-terminal of the fusion protein, and thereby it is also expected that the second Lys of N-terminal can affect the soluble expression of the adhesive protein Mefp1. A leader sequence was determined as from OmpASP fragment (Met[M] and Met-Lys) to the first two amino acids (Ala-Lys) of Mefp1 and the pI value of the leader sequence was analyzed by the computer program DNASIS™ (Hitachi, Japan). As a result, the pI value of Met-Ala-Lys (SEQ. ID. NO: 15) peptide was 9.90, and the pI value of Met-Lys-Ala-Lys (SEQ. ID. NO: 16) was 10.55. To confirm the above result, more clones were constructed by using the coding sequence of a signal sequence fragment OmpASP₁₋₃(Met-Lys-Lys) having one more Lys than OmpASP₁₋₂ by the same manner as described above, followed by quantification of the soluble expression of the adhesive protein Mefp1 (FIG. 1a, line 3). As a result, the soluble expression was related to the controlled pI value of the leader sequence OmpASP₁₋₃(Met-Lys-Lys)-Ala-Lys, which was 10.82 (FIG. 1a, line 3 and Table 1). Therefore, the above result proved that the controlled pI value by Lys in the leader sequence to 9.90-10.82 was related to the soluble expression.

TABLE 1

Primers, leader sequences and the expression of Mefp1
from the pI value increased OmpASPₜᵣ and its variant clones
of recombinant vector pET22b(+) (ompASP₁-7xmefp1*)

| | | | Leader sequence in | |
|---|---|---|---|---|
| | | | which OmpASPₜᵣ and its | |
| | | | variants are linked | |
| SEQ. | | SEQ. | to N-terminal (Ala- | |
| ID. | Forward primer | ID. | Lys) of an adhesive | Soluble |
| NO: | sequence | NO: | protein (pI value) | expression |
| 1 | CAT [symbol] [symbol] [symbol] CCG | 15 | OmpASP₁(Met)-Ala- | ++ |
| | TCT TAT CCG CCA ACC | | Lys(pI 9.90) | |
| | TAC | | | |
| 2 | CAT [symbol] [symbol] [symbol] [symbol] | 16 | OmpASP₁₋₂(Met-Lys)- | +++ |
| | CCG TCT TAT CCG CCA | | Ala-Lys(pI 10.55) | |
| | ACC TAC | | | |
| 3^{A} | CAT [symbol] [symbol] [symbol] [symbol] | 17 | OmpASP₁₋₃(Met-Lys- | +++ |
| | [symbol] CCG TCT TAT CCG | | Lys)-Ala-Lys(pI | |
| | CCA ACC | | 10.82) | |
| 4 | CAT [symbol] [symbol] [symbol] [symbol] | 18 | Met-Lys-Lys-Lys-Ala- | +++ |
| | [symbol] [symbol] CCG TCT TAT | | Lys(pI 10.99) | |
| | CCG CCA ACC | | | |
| 5 | CAT [symbol] [symbol] [symbol] [symbol] | 19 | Met-Lys-Lys-Lys-Lys- | +++ |
| | [symbol] [symbol] [symbol] CCG TCT | | Ala-Lys(pI 11.11) | |
| | TAT CCG CCA ACC | | | |
| 6 | CAT [symbol] [symbol] [symbol] [symbol] | 20 | Met-Lys-Lys-Lys-Lys- | +++ |
| | [symbol] [symbol] [symbol] [symbol] CCG | | Lys-Ala-Lys(pI 11.21) | |
| | TCT TAT CCG CCA ACC | | | |
| 7 | CAT [symbol] [symbol] [symbol] [symbol] | 21 | Met-Lys-Lys-Lys-Lys- | ++ |
| | [symbol] [symbol] [symbol] [symbol] [symbol] | | Lys-Lys-Ala-Lys(pI | |
| | CCG TCT TAT CCG CCA | | 11.28) | |
| | ACC | | | |
| 8 | CAT [symbol] [symbol] [symbol] [symbol] | 22 | Met-Lys-Lys-Lys-Lys- | +/− |
| | [symbol] [symbol] [symbol] [symbol] [symbol] | | Lys-Lys-Lys-Lys-Ala- | |
| | [symbol] [symbol] CCG TCT TAT | | Lys(pI 11.41) | |
| | CCG CCA ACC | | | |
| 9 | CAT [symbol] [symbol] [symbol] [symbol] | 23 | Met-Arg-Ala-Lys(pI | ++ |
| | CCG TCT TAT CCG CCA | | 11.52) | |
| | ACC TAC | | | |
| 10 | CAT [symbol] [symbol] [symbol] [symbol] | 24 | Met-Arg-Arg-Ala- | ++ |
| | [symbol] CCG TCT TAT CCG | | Lys(pI 12.51) | |
| | CCA ACC | | | |
| 11 | CAT [symbol] [symbol] [symbol] [symbol] | 25 | Met-Arg-Arg-Arg-Arg- | ++ |
| | [symbol] [symbol] [symbol] CCG TCT | | Ala-Lys(pI 12.98) | |
| | TAT CCG CCA ACC | | | |
| 12 | CAT [symbol] [symbol] [symbol] [symbol] | 26 | Met-Arg-Arg-Arg-Arg- | ++ |
| | [symbol] [symbol] [symbol] [symbol] [symbol] | | Arg-Arg-Ala-Lys(pI | |
| | CCG TCT TAT CCG CCA | | 13.20) | |
| | ACC | | | |
| 13 | CAT [symbol] [symbol] [symbol] [symbol] | 27 | Met-Arg-Arg-Arg-Arg- | + |
| | [symbol] [symbol] [symbol] [symbol] [symbol] | | Arg-Arg-Arg-Arg-Ala- | |
| | [symbol] [symbol] CCG TCT TAT | | Lys(pI 13.35) | |
| | CCG CCA ACC | | | |

Reverse primer

| | | |
|---|---|---|
| 14^{A} | CTC GAG GTC GAC AAG | — |
| | CTT ACG | |

*Surplus sequence of Ra and His tag (6xHis) shown in FIG. 2 of Korean Patent Publication No. 10-2007-0009453.
CAT: extended for the preservation of NdeI site.
Italic bold letters: oligonucleotides in different sizes encoding the signal sequence fragment-adhesive protein (to the second amino acid of Mefp1: Ala-Lys) and its variants.
General letters: oligonucleotides encoding from the third amino acid of Mefp1 except the first two amino acids Ala-Lys.
( ): The pI value of the leader sequence wherein the signal sequence fragment and its variants are fused to N-terminal (Ala-Lys) of the adhesive protein.
OmpASPₜᵣ: OmpASP fragment described in Korean Patent Publication No. 10-2007-0009453.
^{A}primer constructed in Korean Patent Publication No. 10-2007-0009453.
Reverse primer: Oligonucleotide sequence complementary to Ra (right adapter; Arg/HindIII/SalI/XhoI) shown in FIG. 2 of Korean Patent Publication No. 10-2007-0009453.
As for the expression of the recombinant Mefp1 protein, “−” indicates no expression, “+/−” indicates weak expression and the number of “+” indicates the level of expression.

### Example 4

### Effect of the Increased pI Value of a Leader Sequence on the Expression of an Adhesive Protein

The present inventors confirmed in Example 3 that the control of the pI value of a leader sequence by using Lys was related to the soluble expression of a protein. And the inventors further wanted to confirm whether or not the control of the pI value could affect the general expression of a soluble protein as well. To do so, Lys was additionally inserted in between OmpASP₁₋₃ fragment and Mefp1, resulting in the construction of pET-22b(+)[ompASP₁₋₃-(Lys)ₙ-7×mefp1*] (n=1, 2, 3, 4, 6) (SEQ. ID. NO: 4-SEQ. ID. NO: 8 and SEQ. ID. NO: 18-SEQ. ID. NO: 22), and the amino acid Arg increasing the pI value was also additionally inserted in between Met(OmpASP₁) and Mefp1, resulting in the construction of pET-22b(+)[ompASP₁-(Arg)ₙ-7×mefp1*] (n=1, 2, 4, 6, 8) (SEQ. ID. NO: 9-SEQ. ID. NO: 13 and SEQ. ID. NO: 23-SEQ. ID. NO: 27) (Table 1). The pI value of the leader sequence ranging from the OmpASP fragment to the first two amino acids of Mefp1 (Ala-Lys) of each clone was investigated.

E. coli BL21 (DE3) was transformed with the clone vectors constructed above by the same manner as described in Example 2, and the protein expression induced therein was quantified. As a result, the soluble expression of the adhesive protein Mefp1 fused with the leader sequence having the increased pI value of 10.99-11.21 by the addition of Lys (FIG. 1a, line 4-line 6 and Table 1, SEQ. ID. NO: 18-SEQ. ID. NO: 20) was similar to the level of the control having the pI value of 10.55 (FIG. 1a, line 2 and Table 1, SEQ. ID. NO: 16) or slightly increased. In the meantime, the soluble expression of the adhesive protein Mefp1 fused with the leader sequence having the pI value of 11.28 (FIG. 1a, line 7 and Table 1, SEQ. ID. NO: 21) was reduced, compared with the control having the pI value 10.55. And the soluble expression of the adhesive protein Mefp1 fused with the leader sequence having the pI value of 11.41 (FIG. 1a, line 8 and Table 1, SEQ. ID. NO: 22) was hardly observed. In spite of the increase of the pI value, the leader sequence (SEQ. ID. NO: 22) which having the pI value of 11.41 and exhibiting the reduced expression had comparatively high hydrophilicity (1.93). So, it was presumed that significant increase of hydrophilicity in the leader sequence rather reduced membrane permeability by increasing the binding force to lipid bilayer (Korean Patent Publication No. 10-2007-0009453). However, in spite of high hydrophilicity in the leader sequence (1.14, 1.32, 1.53, and 1.69) having the pI value of 10.99, 11.11, 11.21, and 11.28 respectively, when Lys was additionally inserted, the hydrophilicity was offset to some degree, suggesting that membrane permeation was possible. However, the expression of the adhesive protein Mefp1 had nothing to do with the increase of electric charge.

In addition, of the pI value increased leader sequences (pI 11.52-13.35: SEQ. ID. NO: 23-SEQ. ID. NO: 27), the soluble expression of the adhesive protein Mefp1 fused with the leader sequence having the increased pI value of 11.52-12.51 by the addition of Arg (SEQ. ID. NO: 23-SEQ. ID. NO: 24) was similar to that of the control having the pI value of 9.90 (SEQ. ID. NO: 15) or slightly increased (leader sequence having the pI value of 12.51 by the addition of 2 Args, SEQ. ID. NO: 24), though the increase was not significant. The soluble expression of the adhesive protein Mefp1 fused with the leader sequences having the pI value of 12.98, 13.20 and 13.35 (SEQ. ID. NO: 25-NO: 27) was reduced with the increase of the pI value (Table 1 and FIG. 1b). The leader sequence having the pI value of 13.35 that exhibited the lowest expression (SEQ. ID. NO: 27) had comparatively high hydrophilicity (1.93). So, it was presumed that significant increase of hydrophilicity in the leader sequence rather reduced membrane permeability by increasing the binding force to lipid bilayer (Korean Patent Publication No. 10-2007-0009453). At this time, the expression had nothing to do with the increase of electric charge.

The expression of the adhesive protein Mefp1 fused with the leader sequence having the pI value of 12.51 by the addition of two Args (MRRAK, SEQ. ID. NO: 24) was slightly increased. The soluble adhesive protein Mefp1 having the leader sequence had reduced molecular weight with 3/3 frequencies, suggesting that N-terminal was cut off (FIG. 1b, line 3). This phenomenon was consistently observed in periplasm fractions (data not shown). However, other leader sequences with additional Arg had no deletion. So, the deletion seemed to be attributed to a protease and those leader sequences with additional Arg were expected to have Arg specific membrane permeation mechanism.

### Example 5

### Effect of the Low pI Value of a Leader Sequence on the Soluble Expression of an Adhesive Protein

The present inventors investigated the effect of the down-controlled pI value of N-terminal of a leader sequence on the soluble expression of Mefp1.

Particularly, OmpASP₁-7×mefp1* was used as the control and the amino acid sequence of the leader sequence Met(OmpASP₁)+Ala-Lys (N-terminal of Mefp1) was differently modified to produce variants of the leader sequence represented by SEQ. ID. NO: 35-SEQ. ID. NO: 41 [MDDDDDAA (SEQ. ID. NO: 35; pI=2.73), MDDDAA (SEQ. ID. NO: 36; pI=2.87), MEE (SEQ. ID. NO: 37; pI=3.09), MAE (SEQ. ID. NO: 38; pI=3.25), MAA (SEQ. ID. NO: 39; pI=5.60), MCH (SEQ. ID. NO: 40; pI=7.13), MAH (SEQ. ID. NO: 41; pI=7.65)] having the pI value of 2.73-7.65 (Table 2). The pI values of those variants were investigated. MAK (SEQ. ID. NO: 15; pI=9.90) and MRRRRAK (SEQ. ID. NO: 25; pI=12.98) were used as the controls and the expressions were investigated.

E. coli BL21 (DE3) was transformed with the clone vectors constructed above by the same manner as described in Example 2, and the protein expression therein was quantified. As a result, the soluble adhesive protein Mefp1 expression was observed in every clone containing the leader sequences represented by SEQ. ID. NO: 35-SEQ. ID. NO: 41. Particularly, the clones containing the leader sequences having the pI values of 3.09-7.65 (SEQ. ID. NO: 37-SEQ. ID. NO: 41) exhibited significantly higher expression than those in the clones containing the leader sequences having the pI values of 9.90 (SEQ. ID. NO: 15) and 12.98 (SEQ. ID. NO: 25), and especially higher expression was observed when the pI value was controlled to 3.09 (SEQ. ID. NO: 37) (FIG. 2 and Table 2). Even the leader sequence exhibiting the lowest expression (SEQ. ID. NO: 35; pI=2.73) had comparatively high hydrophilicity of 1.09. So, it was presumed that significant increase of hydrophilicity in the leader sequence rather reduced membrane permeability by increasing the binding force to lipid bilayer (Korean Patent Publication No. 10-2007-0009453). At this time, the expression had nothing to do with the increase of electric charge.

TABLE 2

Expressions of variant clones of the leader sequence
(Met-Ala-Lys) of recombinant vector pET22b(+) ompASP₁-7xmefp1*

| | | | Leader sequence with | |
|---|---|---|---|---|
| SEQ. | | SEQ. | modified | |
| ID. | Forward primer | ID. | OmpASP₁(Met)-Ala-Lys | Soluble |
| NO: | sequence | NO: | (pI value) | expression |
| 28 | CAT [symbol] [symbol] [symbol] [symbol] | 35 | Met-Asp-Asp-Asp-Asp- | + |
| | [symbol] [symbol] [symbol] [symbol] CCG | | Asp-Ala-Ala | |
| | TCT TAT CCG CCA ACC | | (pI 2.73) | |
| | TAC | | | |
| 29 | CAT [symbol] [symbol] [symbol] [symbol] | 36 | Met-Asp-Asp-Asp-Ala- | ++ |
| | [symbol] [symbol] CCG TCT TAT | | Ala(pI 2.87) | |
| | CCG CCA ACC TAC | | | |
| 30 | CAT [symbol] [symbol] [symbol] CCG | 37 | Met-Glu-Glu(pI 3.09) | +++ |
| | TCT TAT CCG CCA ACC | | | |
| | TAC | | | |
| 31 | CAT [symbol] [symbol] [symbol] CCG | 38 | Met-Ala-Glu(pI 3.25) | +++ |
| | TCT TAT CCG CCA ACC | | | |
| | TAC | | | |
| 32 | CAT [symbol] [symbol] [symbol] CCG | 39 | Met-Ala-Ala(pI 5.60) | +++ |
| | TCT TAT CCG CCA ACC | | | |
| | TAC | | | |
| 33 | CAT [symbol] [symbol] [symbol] CCG | 40 | Met-Cys-His(pI 7.13) | +++ |
| | TCT TAT CCG CCA ACC | | | |
| | TAC | | | |
| 34 | CAT [symbol] [symbol] [symbol] CCG | 41 | Met-Ala-His(pI 7.65) | +++ |
| | TCT TAT CCG CCA ACC | | | |
| | TAC | | | |

Reverse primer

| | | |
|---|---|---|
| 14^{A} | CTC GAG GTC GAC AAG | — |
| | CTT ACG | |

*Surplus sequence of Ra and His tag (6xHis) shown in FIG. 2 of Korean Patent Publication No. 10-2007-0009453.
CAT: extended for the preservation of NdeI site.
Italic bold letters: oligonucleotides in different sizes encoding the leader sequence (Met-Ala-Lys) and its variants.
General letters: oligonucleotides encoding from the third amino acid of Mefp1 except the first two amino acids Ala-Lys.
( ): The pI value of the leader sequence (Met-Ala-Lys) variant.
^{A}primer constructed in Korean Patent Publication No. 10-2007-0009453.
Reverse primer: Oligonucleotide sequence complementary to Ra (right adaptor; Arg/HindIII/SalI/XhoI) shown in FIG. 2 of Korean Patent Publication No. 10-2007-0009453.
As for the expression of the recombinant Mefp1 protein, “−” indicates no expression, “+/−” indicates weak expression and the number of “+” indicates the level of expression.

### Example 6

### Optimization of the Distance Between a Leader Sequence and a Factor Xa Recognition Site (Xa) for the Production of an Adhesive Protein in Native Form

In Example 5, from the investigation of the expression patterns of Mefp1 protein, which resulted in the increase of the expression by the controlled pI value of the leader sequence, it was confirmed that one of the optimum pI value of the leader sequence of the adhesive foreign protein Mefp1 was 3.09 (MEE; SEQ. ID. NO: 37). Then, the distance between the leader sequence having the pI value of 3.09 (MEE) and the Xa factor recognition site (Xa) was optimized by controlling the distance between the leader sequence and Mefp1 sequence linked thereto, followed by production of a fusion protein facilitating the recovery of a soluble protein having the native amino terminal according to the method described in Korean Patent Publication No. 10-2007-0009453. The structural change resulted from the extension of the leader sequence was minimized by using some parts (Mefp1₃₋₈) of amino acids of Mefp1 linked to the leader sequence (MEE) as an insert (i).

Particularly, the factor Xa recognition site (Xa) was included, resulting in MEE-(i=n)-Xa, and amino acids of a part of Mefp1 linked to the leader sequence MEE, which is presented as n, were inserted (n=0, 2, 4, and 6) to construct the clone pET-22b(+)(MEE-(i=n)-Xa-7×mefp1*) for the optimum protein expression (Table 3).

E. coli BL21 (DE3) was transformed with the clone vector constructed above by the same manner as described in Example 2, and the protein expression was quantified. As a result, the expression was most significantly reduced when the distance between MEE and Xa was 4, precisely in the order of i=0>2>6>4 (FIG. 3). The soluble protein included the factor Xa recognition site (Xa), so that the recombinant protein having native N-terminal (7×Mefp1*) with the elimination of MEE-(i=n)-Xa could be produced by the conventional method after treating the recombinant protein with factor Xa protease.

TABLE 3

Expression of the recombinant vector pET-22b(+) (MEE-
(i = n)-Xa-7xmefp1*)

| | | | | |
|---|---|---|---|---|
| | | | Amino acid sequence | |
| | | | (pI 4.01) of leader | |
| SEQ. | | SEQ. | sequence and the | |
| ID. | Forward primer | ID. | number of inserted | Soluble |
| NO: | sequence | NO: | amino acid (i) | expression |

| | | | | |
|---|---|---|---|---|
| 42 | CAT ATG GAA GAG ATC | 46 | Met-Glu-Glu-Xa(i = 0) | +++ |
| | GAA GGT CGT GCT AAG | | | |
| | CCG TCT TAT CCG CCA | | | |
| | ACC TAC | | | |
| 43 | CAT ATG GAA GAG [symbol] | 47 | Met-Glu-Glu-Pro-Ser- | +++ |
| | [symbol] ATC GAA GGT CGT | | Xa(i = 2) | |
| | GCT AAG CCG TCT TAT | | | |
| | CCG CCA ACC TAC | | | |
| 44 | CAT ATG GAA GAG [symbol] | 48 | Met-Glu-Glu-Pro-Ser- | + |
| | [symbol] [symbol] [symbol] ATC GAA | | Tyr-Pro-Xa(i = 4) | |
| | GGT CGT GCT AAG CCG | | | |
| | TCT TAT CCG CCA ACC | | | |
| | TAC | | | |
| 45 | CAT ATG GAA GAG [symbol] | 49 | Met-Glu-Glu-Pro-Ser- | ++ |
| | [symbol] [symbol] [symbol] [symbol] [symbol] | | Tyr-Pro-Pro-Thr- | |
| | ATC GAA GGT CGT GCT | | Xa(i = 6) | |
| | AAG CCG TCT TAT CCG | | | |
| | CCA ACC TAC | | | |

Reverse primer

| | |
|---|---|
| 14^{A} | CTC GAG GTC GAC AAG |
| | CTT ACG |

*Surplus sequence of Ra and His tag (6xHis) shown in FIG. 2 of Korean Patent Publication No. 10-2007-0009453.
CAT: extended for the preservation of NdeI site.
Bold letters: oligonucleotide of the leader sequence(MEE).
Italic bold letters: oligonucleotide of a part of Mefp1 (Mefp1₃₋₈) linked to the leader sequence (MEE) of Table 2.
ATC GAA GGT CGT: oligonucleotide of the Xa factor recognition site.
General letters: oligonucleotide encoding the basic amino acid sequence of Mefp 1 represented by SEQ. ID. NO: 1 described in Korean Patent Publication No. 10-2007-0009453.
^{A}primer constructed in Korean Patent Publication No. 10-2007-0009453.
Reverse primer: Oligonucleotide sequence complementary to Ra (right adaptor; Arg/HindIII/SalI/XhoI) shown in FIG. 2 of Korean Patent Publication No. 10-2007-0009453.
As for the expression of the recombinant Mefp1 protein, “−” indicates no expression, “+/−” indicates weak expression and the number of “+” indicates the level of expression.
(i): amino acid number inserted in between the leader sequence (MEE) and Xa.

### Example 7

### Soluble Expression of an Adhesive Protein by the Control of the Distance Between Lys-Lys in OmpASP₁₋₁₁

The present inventors confirmed that the pI value of N-terminal containing a signal sequence could affect the soluble expression of an adhesive foreign protein Mefp1. Then, the inventors further investigated if the distance between amino acids (for example between Lys-Lys) affecting the pI value in OmpA signal sequence fragment (OmpASPₜᵣ) could affect the soluble expression of Mefp1. Particularly, the leader sequences MKK (SEQ. ID. NO: 56) and MKAK (SEQ. ID. NO: 16) had the equal pI value of 10.55 in N-terminal, but if the distance (d) between Lys-Lys was farther because of the insertion of the amino acid less affecting the pI value (for example, Ala [Alanine; A]), there might be changes in functions.

Based on the amino acid sequence of the signal sequence fragment OmpASP₁₋₁₁, pET-22b(+)[MK₁-(d₁=n)-K₂-(8-n)-AA-mefp1₃₋₁₀-6×mefp1*] was constructed by inserting OmpASP₁₋₁₁ composing amino acids d₁=n(0, 2, 4, 6, 8) not affecting the pI value into the ( ) which was designed as MK₁-(d=n)-K₂-(8-n) analogue (Table 4). The leader sequence of the above clone had the equal pI value of 10.55 from OmpASP₁₋₂ fragment (Met-Lys) to the underlined second Ala (Ala-Ala) taking the place of the second Lys of Mefp1 affecting the pI value. E. coli BL21 (DE3) was transformed with the clone constructed above as shown in Table 4 by the same manner as described in Example 2 and the protein expression therein was quantified.

As a result, the expression was reduced in the order of d₁=4>2>6>0>8 (d₁=distance of K₁-K₂). That is, when d₁=4, the soluble expression was most significant so that d₁=4 was determined as the optimum distance between amino acids. Additionally, d₁ was regulated as 4 and the underlined Ala of the clone was substituted with Lys (K₃) and Ala was inserted with d₂=n(1, 2, 3, 4), resulting in the construction of the clone pET-22b(+)[MK₁-(d₁=4)-K₂-(d₂=n)-AK₃-mefp1₃₋₁₀-6×mefp1*] (Table 4), followed by quantification of the protein expression by the same manner as described above.

The optimum distance between two amino acids (K₂-K₃) was d₂=2 and d₂=2>1>4>3 followed in that order. This result indicates that the optimum distance is also an important factor affecting the soluble expression of the adhesive protein Mefp1.

It was also suggested that the important factor is the distance between Lys-Lys and the pI value of the leader sequence not the sequence itself (Table 4 and FIG. 4).

In conclusion, the pI value of the leader sequence played an important role in the soluble expression of the adhesive protein Mefp1 and there was the optimum pI value in its spectrum for the best expression. However, the soluble expression of the adhesive protein Mefp1 had nothing to do with electric charge. The distance between Lys and Lys affecting the pI value was also an important factor for the expression.

TABLE 4

Primers, leader sequence and the expression of Mefp1
from OmpASP₁₋₁₁ variant clones of pET22b(+)
ompASP₁₋₁₁-7xmefp1*

| | | | | |
|---|---|---|---|---|
| | | | Amino acid sequence | |
| | | | shows the leader | |
| | | | sequence in which | |
| | | | OmpASP₁₋₁₁'s variants | |
| | | | are linked to N- | |
| | | | terminal (Ala-Ala) of | |
| | | | an adhesive protein | |
| SEQ. | | SEQ. | (pI 10.55 and 10.82) | |
| ID. | Forward primer | ID. | and the distance (d) | Soluble |
| NO: | sequence | NO: | between Lys. | expression |

| | | | | |
|---|---|---|---|---|
| 50^{A} | CAT ATG [symbol] [symbol] | 56 | Met-Lys-Lys-Thr-Ala- | positive |
| | ACA GCT ATC GCG | | Ile-Ala-Ile-Ala-Val- | control, |
| | ATT GCA GTG GCA | | Ala-Ala-Lys(pI 10.82) | +++ |
| | GCT [symbol] CCG TCT | | | |
| | TAT CCG CCA ACC | | | |
| | TAC | | | |
| 51 | CAT ATG [symbol] [symbol] | 57 | Met-Lys-Lys-Thr-Ala- | Ala |
| | ACA GCT ATC GCG | | Ile-Ala-Ile-Ala-Val- | mutant |
| | ATT GCA GTG GCA | | Ala-Ala-Ala(pI 10.55, | control, |
| | GCT GCA CCG TCT | | d₁ = 0) | ++ |
| | TAT CCG CCA ACC | | | |
| | TAC | | | |
| 52 | CAT ATG [symbol] GCT | 58 | Met-Lys-Ala-Thr-Lys- | +++ |
| | ACA [symbol] ATC GCG | | Ile-Ala-Ile-Ala-Val- | |
| | ATT GCA GTG GCA | | Ala-Ala-Ala(pI 10.55, | |
| | GCT GCA CCG TCT | | d₁ = 2) | |
| | TAT CCG CCA ACC | | | |
| | TAC | | | |
| 53 | CAT ATG [symbol] GCT | 59 | Met-Lys-Ala-Thr-Ala- | +++ |
| | ACA GCT ATC [symbol] | | Ile-Lys-Ile-Ala-Val- | |
| | ATT GCA GTG GCA | | Ala-Ala-Ala(pI 10.55, | |
| | GCT GCA CCG TCT | | d₁ = 4) | |
| | TAT CCG CCA ACC | | | |
| | TAC | | | |
| 54 | CAT ATG [symbol] GCT | 60 | Met-Lys-Ala-Thr-Ala- | ++ |
| | ACA GCT ATC GCG | | Ile-Ala-Ile-Lys-Val- | |
| | ATT [symbol] GTG GCA | | Ala-Ala-Ala(pI 10.55, | |
| | GCT GCA CCG TCT | | d₁ = 6) | |
| | TAT CCG CCA ACC | | | |
| | TAC | | | |
| 55 | CAT ATG [symbol] GCT | 61 | Met-Lys-Ala-Thr-Ala- | + |
| | ACA GCT ATC GCG | | Ile-Ala-Ile-Ala-Val- | |
| | ATT GCA GTG [symbol] | | Lys-Ala-Ala(pI 10.55, | |
| | GCT GCA CCG TCT | | d₁ = 8) | |
| | TAT CCG CCA ACC | | | |
| | TAC | | | |
| 102 | CAT ATG [symbol] GCT | 106 | Met-Lys-Ala-Thr-Ala- | +++ |
| | ACA GCT ATC [symbol] | | Ile-Lys-Ala-Lys(pI | |
| | GCT [symbol] CCG TCT | | 10.82, d₁ = 4, d₂ = 1) | |
| | TAT CCG CCA ACC | | | |
| | TAC | | | |
| 103 | CAT ATG [symbol] GCT | 107 | Met-Lys-Ala-Thr-Ala- | ++++ |
| | ACA GCT ATC [symbol] | | Ile-Lys-Ala-Ala- | |
| | GCA GCT [symbol] CCG | | Lys(pI 10.82, d₁ = 4, | |
| | TCT TAT CCG CCA | | d₂ = 2); | |
| | ACC TAC | | | |
| 104 | CAT ATG [symbol] GCT | 108 | Met-Lys-Ala-Thr-Ala- | ++ |
| | ACA GCT ATC [symbol] | | Ile-Lys-Ala-Ala-Ala- | |
| | GCT GCA GCT [symbol] | | Lys(pI 10.82, d₁ = 4, | |
| | CCG TCT TAT CCG | | d₂ = 3); | |
| | CCA ACC TAC | | | |
| 105 | CAT ATG [symbol] GCT | 109 | Met-Lys-Ala-Thr-Ala- | +++ |
| | ACA GCT ATC [symbol] | | Ile-Lys-Ala-Ala-Ala- | |
| | GCA GCT GCA GCT | | Ala-Lys(pI 10.82, | |
| | [symbol] CCG TCT TAT | | d₁ = 4, d₂ = 4); | |
| | CCG CCA ACC TAC | | | |

Reverse primer

| | |
|---|---|
| 14^{A} | CTC GAG GTC GAC |
| | AAG CTT ACG |

*Surplus sequence of Ra and His tag (6xHis) shown in FIG. 2 of Korean Patent Publication No. 10-2007-0009453.
CAT: extended for the preservation of NdeI site.
Italic letters: oligonucleotide encoding the leader sequence of the signal sequence fragment OmpASP₁₋₁₁ or its variants linked to N-terminal (Ala-Ala) of an adhesive protein.
Italic bold letters: oligonucleotide encoding the amino acid lys in the leader sequence fragment.
Ala: Lys of Ala-Lys of N-terminal of an adhesive protein was substituted with Ala.
General letters: oligonucleotide encoding from the third amino acid of Mefp1 except the first two amino acids Ala-Lys.
^{A}primer constructed in Korean Patent Publication No. 10-2007-0009453.
Reverse primer: Oligonucleotide sequence complementary to Ra (right adaptor; Arg/HindIII/SalI/XhoI) shown in FIG. 2 of Korean Patent Publication No. 10-2007-0009453.
As for the expression of the recombinant Mefp1 protein, “−” indicates no expression, and the number of “+” indicates the level of expression.
(d): distance between Lys-Lys.

### Example 8

### Soluble Expression of Olive Flounder Hepcidin I by N-Terminal Variants

Based on the earlier experiment results reported in Korean Patent Publication No. 10-2007-009453 saying that the soluble expression of Hepcidin I (Kim et al., Biosci Biotechnol Biochem 69:1411-1414, 2005) requires a signal sequence and a secretional enhancer, the present inventors constructed a recombinant vector for the soluble expression of Hepcidin I with controlling the pI value of N-terminal of the leader sequence of Hepcidin I. Particularly, a leader sequence functioning as a signal sequence and at the same time as a secretional enhancer or a signal sequence OmpASP fragment variant, a secretional enhancer candidate sequence or/and Xa recognition site were operably linked to ofHep1, which was introduced into pET-22b(+) (Table 5 and Table 6).

E. coli BL21 (DE3) was transformed with the expression vector containing N-terminal constructed as shown in Table 5 and Table 6, followed by culture in LB medium (tryptone 10 g, yeast extract 5 g, NaCl 10 g/l) supplemented with 50 μg/ml of ampicillin at 30° C. for 16 hours. The culture solution was diluted 200 times with the LB medium. 1 mM of IPTG was added to the diluted culture solution, followed by culture until OD₆₀₀ reached 0.3. The culture continued for 3 hours to induce the expression. 1 ml of the culture solution proceeded to centrifugation at 4° C., 4,000×g for 30 minutes and the pellet was resuspended in 100-200 μl of PBS. The suspension was homogenized to separate a protein by using a sonicator at 15× 2-s cycle pulses (at 30% power output). Centrifugation was performed at 4° C., 16,000 rpm for 30 minutes to eliminate cell debris, resulting in the separation of an insoluble fraction. The protein of a soluble fraction was quantified by Bradford method (Bradford, Anal Biochem 72:248-254, 1976), followed by SDS-PAGE by using 15% SDS-PAGE gel according to the method of Laemmli et al (Laemmli, Nature 227:680-685, 1970). Coomassie Brilliant Blue (Sigma, USA) staining was performed. The SDS-PAGE gel was transferred onto a nitrocellulose membrane (Roche, USA). After dipping in 5% skim milk (skimmed milk; Difco, USA), the membrane was soaked in 0.4 μg/ml of anti-His6 monoclonal antibody solution (Santa Cruz Biotechnology, USA) at 37° C. for 2 hours. DAB (3,3′-diaminobenzidine tetrahydrochloride, Sigma, USA) staining was performed using horseradish peroxidase conjugated rabbit anti-mouse IgG (Santa Cruz Biotechnology, USA) as a secondary antibody.

### Example 9

### Soluble Expression of Olive Flounder Hepcidin I by Controlling the pI Value of a Leader Sequence

The present inventors investigated the effect of pI control in a leader sequence on the soluble expression of olive flounder Hepcidin I by the similar manner as described in Example 4 and Example 5.

For the soluble expression of olive flounder Hepcidin I, similarly to the screening method of a secretional enhancer described in Korean Patent Publication No. 10-2007-0009453, pET-22b(+)(Met-7×homologous amino acids-ofhep I**) clone was constructed for the expression of the protein having the controlled pI value of 2.52-13.28 and the hydrophobicity/hydrophilicity of −1.55-+1.97 which was designed to connect N-terminal of the protein to Met-7×homologous amino acids to contain Met and a secretional enhancer candidate sequence (Table 5). The homologous amino acid herein was selected from the group consisting of arginine (Arg; R), lysine (Lys, K), histidine (His; H), tyrosine (Tyr; Y), cysteine (Cys; C), glutamic acid (Glu; E) and aspartic acid (Asp; D), which was supposed to have repeats. The hydrophobicity was measured by DNASIS™ (Hitachi, Japan) as Hopp & Woods scale (window size: 6, threshold: 0.00). If the hydrophobicity value is +, the peptide is hydrophilic, while if the hydrophobicity value is −, the peptide is hydrophobic. And, as the value increases, hydrophilicity or hydrophobicity increases.

E. coli BL21 (DE3) was transformed with the clone vector constructed above by the same manner as described in Example 8, followed by quantification of the protein expression. As a result, the soluble expression of Hepcidin I was observed only in the clones having MRRRRRRR (pI: 13.28, hydrophobicity: +1.97 [hydrophilic]) and MKKKKKKK (pI: 11.28, hydrophobicity: +1.97 [hydrophilic]) (FIG. 5).

TABLE 5

Expression of the recombinant vector pET-22b(+) (Met-
7xhomologous aas-ofhep I**)

| | | | Amino acid | |
|---|---|---|---|---|
| | | | sequence (pI | |
| SEQ. | | SEQ. | value, | |
| ID. | Forward primer | ID. | hydrophobicity | Soluble |
| NO: | sequence | NO: | value hy) | expression |
| 62 | CAT ATG CGT CGC CGT | 70 | MRRRRRRR(pI 13.28, | ++ |
| | CGC CGT CGC CGT CAC | | hy +1.97) | |
| | ATC AGC CAC ATC TCC | | | |
| | ATG TGC | | | |
| 63 | CAT ATG AAA AAA AAA | 71 | MKKKKKKK(pI 11.28, | ++ |
| | AAA AAA AAA AAA CAC | | hy +1.97) | |
| | ATC AGC CAC ATC TCC | | | |
| | ATG TGC | | | |
| 64 | CAT ATG CAC CAT CAC | 72 | MHHHHHHH(pI 8.08, | − |
| | CAT CAC CAT CAC CAC | | hy −0.35) | |
| | ATC AGC CAC ATC TCC | | | |
| | ATG TGC | | | |
| 65 | CAT ATG TAC TAT TAC | 73 | MYYYYYYY(pI 5.59, | − |
| | TAT TAC TAT TAC CAC | | hy −1.55) | |
| | ATC AGC CAC ATC TCC | | | |
| | ATG TGC | | | |
| 66 | CAT ATG TGC TGT TGC | 74 | MCCCCCCC(pI 4.57, | − |
| | TGT TGC TGT TGC CAC | | hy −0.69) | |
| | ATC AGC CAC ATC TCC | | | |
| | ATG TGC | | | |
| 67 | CAT ATG GAA GAG GAA | 75 | MEEEEEEE(pI 2.78, | − |
| | GAG GAA GAG GAA CAC | | hy +1.97) | |
| | ATC AGC CAC ATC TCC | | | |
| | ATG TGC | | | |
| 68 | CAT ATG GAC GAT GAC | 76 | MDDDDDDD(pI 2.52, | − |
| | GAT GAC GAT GAC CAC | | hy +1.97) | |
| | ATC AGC CAC ATC TCC | | | |
| | ATG TGC | | | |

Reverse primer

| | |
|---|---|
| 69^{A} | CTC GAG GTC GAC AAG CTT |
| | TTC GAA CTT GCA GCA GGG |
| | GCC ACA GCC |

CAT: extended for the preservation of NdeI site.
Bold letters: oligonucleotides having different sizes of the leader sequences affecting the pI value and the hydrophobicity.
ofhepI: olive flounder Hepcidin I (ofHepcidinI: ofHepI) gene (Korean Patent Publication No. 10-2007-0009453; Kim et al., Biosci. Biotechnol. Biochem. 69, 1411-1414, 2005).
**Glu/HindIII/SalI/XhoI-6xHis described in Korean Patent Publication No. 10-2007-0009453 (Glu/HindIII/SalI/XhoI is originated from the reverse primer design).
General letters: oligonucleotide of Hepcidin I region.
^{A}primer constructed in Korean Patent Publication No. 10-2007-0009453.
Reverse primer: Oligonucleotide sequence containing C-terminal and Glu/HindIII/SalI/XhoI site of ofHepcidinI of Korean Patent Publication No. 10-2007-0009453.
As for the expression of the recombinant ofHep I**, “−” indicates no expression, and the number of “+” indicates the level of expression.
Hydrophobicity: calculated by DNASIS ™(Hitachi, Japan) as Hopp & Woods scale (window size: 6, threshold: 0.00). If the hydrophobicity value is +, the peptide is hydrophilic, while if the hydrophobicity value is −, the peptide is hydrophobic. And, as the absolute value increases, hydrophilicity or hydrophobicity increases.

### Example 10

### Effect of the Low pI Value of the Signal Sequence Variants on the Soluble Expression of Olive Flounder Hepcidin I

Korean Patent Publication No. 10-2007-009453 describes the soluble expression of olive flounder Hepcidin I by using OmpASP₁₋₁₀ having the comparatively high pI value of 10.55 as a signal sequence. However, the present inventors confirmed in Example 4 and Example 5 of the invention that not only the high pI value but also the low pI value of the leader sequence could increase the soluble expression of an adhesive protein Mefp1. Thus, the present inventors investigated the effect of the low pI value of the signal sequence variants on the soluble expression of olive flounder Hepcidin I.

Particularly, a leader sequence was designed to contain the signal sequence fragment OmpASP₁₋₃ variants [MAH (SEQ. ID. NO: 41); 7.65, MAA (SEQ. ID. NO: 39); 5.60 or MEE (SEQ. ID. NO: 37); 3.09]-OmpASP₄₋₁₀-6×homologous amino acids (amino acids having the different pI values and the hydrophobicity values)-Xa recognition site (Xa) in that order, from which a clone for the expression of Hepcidin I was constructed (Table 6). The homologous amino acid herein was selected from the group consisting of arginine (Arg; R), tyrosine (Tyr; Y) and glutamic acid (Glu; E), which was supposed to have 6 repeats. The hydrophobicity was calculated by DNASIS™ (Hitachi, Japan) as Hopp & Woods scale (window size: 6, threshold: 0.00). If the hydrophobicity value is +, the peptide is hydrophilic, while if the hydrophobicity value is −, the peptide is hydrophobic. And, as the absolute value increases, hydrophilicity or hydrophobicity increases.

E. coli BL21 (DE3) was transformed with the clone vector of Table 6 by the same manner as described in Example 8, followed by quantification of the protein expression. As a result, the recombinant protein MAH(pI 7.65)-OmpASP₄₋₁₀-6×Arg-Xa-ofHep I** was well expressed. MAH(pI 7.65) -OmpASP₄₋₁₀-6×Glu-Xa-ofHep I ** was also expressed but very weakly. MAA(pI 5.60)-OmpASP₄₋₁₀-6×Arg-Xa-ofHep I** was well expressed. MAA (pI 5.60)-OmpASP₄₋₁₀-6×Glu-Xa-ofHep I** was weakly expressed. MEE(pI 3.09)-OmpASP₄₋₁₀-6×Arg-Xa-ofHep I** was expressed and MEE(pI 3.09) -OmpASP₄₋₁₀-6×Glu-Xa-ofHep I** was also well expressed (FIG. 6).

The above results indicate that when a secretional enhancer sequence is directly linked to Met, the soluble expression is limited to Arg and Lys having the high pI value and hydrophilicity. However, when the pI value of N-terminal of the signal sequence fragment having hydrophobic region is changed, the spectrum of a secretional enhancer is broadened so that not only amino acids having the high pI value and high hydrophilicity but also amino acids having the low pI value and high hydrophilicity such as Glu can be used as a secretional enhancer sequence. Therefore, it is suggested that the hydrophobic region linked to N-terminal of the leader sequence reduces the original hydrophilicity of N-terminal so as to make the fragment act as a free anchor functioning as a signal sequence, resulting in broadening the spectrum of a secretional enhancer. Based on the above results, it is also expected that the pI value of N-terminal of the leader sequence has inter-relationship with the secretional enhancer sequence, as confirmed in the above as the controlled pI value of N-terminal of the signal sequence results in the change of the soluble expression.

TABLE 6

Expression of the recombinant vector pET-
22b(+)(ompASP₁-2 aas-ompASP₄-10-6xhomologous
aas-Xa-ofhepI**)

| | | | Signal sequence with | |
|---|---|---|---|---|
| | | | the low pI value + | |
| | | | secretional enhancer | |
| | | | candidate sequence | |
| SEQ. | | SEQ. | (pI value and/or | |
| ID. | | ID. | hydrophobicity | Soluble |
| NO: | Forward primer sequence | NO: | value) | expression |
| 77 | CAT ATG GCT CAC ACA GCT | 86 | MAH(pI 7.65)-TAI AIA | +++ |
| | ATC GCG ATT GCA GTG [symbol] | | V(OmpASP₄₋₁₀, pI | |
| | [symbol] [symbol] [symbol] [symbol] [symbol] (ATC | | 5.70)-6xArg(pI | |
| | GAA GGT CGT) CAC ATC | | 13.20; hy 1.75)- | |
| | AGC CAC ATC TCC ATG TGC | | Xa(pI 7.05) | |
| 78 | CAT ATG GCT CAC ACA GCT | 87 | MAH-OmpASP₄₋₁₀-6xTyr | − |
| | ATC GCG ATT GCA GTG [symbol] | | (pI 5.55; hy −1.33)- | |
| | [symbol] [symbol] [symbol] [symbol] [symbol] (ATC | | Xa | |
| | GAA GGT CGT) CAC ATC | | | |
| | AGC CAC ATC TCC ATG TGC | | | |
| 79 | CAT ATG GCT CAC ACA GCT | 88 | MAH-OmpASP₄₋₁₀-6xGlu | +/− |
| | ATC GCG ATT GCA GTG [symbol] | | (pI 2.82; hy 1.75)- | |
| | [symbol] [symbol] [symbol] [symbol] [symbol] (ATC | | Xa | |
| | GAA GGT CGT) CAC ATC | | | |
| | AGC CAC ATC TCC ATG TGC | | | |
| 80 | CAT ATG GCT GCA ACA GCT | 89 | MAA(pI 5.60)-OmpASP₄₋₁₀- | +++ |
| | ATC GCG ATT GCA GTG [symbol] | | 6xArg-Xa | |
| | [symbol] [symbol] [symbol] [symbol] [symbol] (ATC | | | |
| | GAA GGT CGT) CAC ATC | | | |
| | AGC CAC ATC TCC ATG TGC | | | |
| 81 | CAT ATG GCT GCA ACA GCT | 90 | MAA-OmpASP₄₋₁₀-6xTyr- | − |
| | ATC GCG ATT GCA GTG [symbol] | | Xa | |
| | [symbol] [symbol] [symbol] [symbol] [symbol] (ATC | | | |
| | GAA GGT CGT) CAC ATC | | | |
| | AGC CAC ATC TCC ATG TGC | | | |
| 82 | CAT ATG GCT GCA ACA GCT | 91 | MAA-OmpASP₄₋₁₀-6xGlu- | +/− |
| | ATC GCG ATT GCA GTG [symbol] | | Xa | |
| | [symbol] [symbol] [symbol] [symbol] [symbol] (ATC | | | |
| | GAA GGT CGT) CAC ATC | | | |
| | AGC CAC ATC TCC ATG TGC | | | |
| 83 | CAT ATG GAA GAG ACA GCT | 92 | MEE(pI 3.09)-OmpASP₄₋₁₀- | + |
| | ATC GCG ATT GCA GTG [symbol] | | 6xArg-Xa | |
| | [symbol] [symbol] [symbol] [symbol] [symbol] (ATC | | | |
| | GAA GGT CGT) CAC ATC | | | |
| | AGC CAC ATC TCC ATG TGC | | | |
| 84 | CAT ATG GAA GAG ACA GCT | 93 | MEE-OmpASP₄₋₁₀-6xTyr- | − |
| | ATC GCG ATT GCA GTG [symbol] | | Xa | |
| | [symbol] [symbol] [symbol] [symbol] [symbol] (ATC | | | |
| | GAA GGT CGT) CAC ATC | | | |
| | AGC CAC ATC TCC ATG TGC | | | |
| 85 | CAT ATG GAA GAG ACA GCT | 94 | MEE-OmpASP₄₋₁₀-6xGlu- | ++ |
| | ATC GCG ATT GCA GTG [symbol] | | Xa | |
| | [symbol] [symbol] [symbol] [symbol] [symbol] (ATC | | | |
| | GAA GGT CGT) CAC ATC | | | |
| | AGC CAC ATC TCC ATG TGC | | | |

Reverse primer

| | |
|---|---|
| 69^{A} | CTC GAG GTC GAC AAG CTT |
| | TTC GAA CTT GCA GCA GGG |
| | GCC ACA GCC |

ofhepI: olive flounder Hepcidin I (of HepcidinI) gene (Kim et al., Biosci Biotechnol Biochem 69: 1411-1414, 2005).
**Glu/HindIII/SalI/XhoI-6xHis described in Korean Patent Publication No. 10-2007-0009453 (Glu/HindIII/SalI/XhoI is originated from the reverse primer design).
CAT: extended for the preservation of NdeI site.
Bold letters: oligonucleotides of the signal sequence variants affecting the pI value.
General letters: oligonucleotides of the OmpASP₄₋₁₀.
Italic bold letters: oligonucleotides of amino acids related with the different pI values and the hydrophobicity values, which is the secretional enhancer candidate sequences.
Underlined plain letters: oligonucleotides of the factor Xa recognition site.
Italic letters: oligonucleotide of Hepcidin I region (Korean Patent Publication No. 10-2007-0009453; Kim et al., Biosci Biotechnol Biochem 69: 1411-1414, 2005).
^{A}primer constructed in Korean Patent Publication No. 10-2007-0009453.
Reverse primer: oligonucleotide sequence containing C-terminal and Glu/HindIII/SalI/XhoI site of of HepcidinI of Korean Patent Publication No. 10-2007-0009453.
As for the expression of the recombinant of HepI**, “−” indicates no expression, “+/−” indicates weak expression and the number of “+” indicates the level of expression.
Hydrophobicity: calculated by DNASIS ™(Hitachi, Japan) as Hopp & Woods scale (window size: 6, threshold line: 0.00). If the hydrophobicity value is +, the peptide is hydrophilic, while if the hydrophobicity value is −, the peptide is hydrophobic. And, as the absolute value increases, hydrophilicity or hydrophobicity increases.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An expression vector for improving secretion efficiency of a foreign protein containing a gene construct which comprises:
(i) a promoter; and
(ii) a polynucleotide operably linked to the promoter encoding a polypeptide fragment containing a signal sequence and/or pI value of the N-region of the leader sequence of a foreign protein and/or the leader sequence or variants thereof in which the distance between amino acids affecting the pI value is controlled.

2. The expression vector according to claim 1, wherein the polypeptide fragment containing N-region with the controlled pI value is the polypeptide composed of 1-6 amino acids where the pI value is controlled to 9.90-11.41 or the polypeptide composed of 1-12 amino acids where the pI value is controlled to 3.09-9.89.

3-10. (canceled)

11. An expression vector for improving secretion efficiency of a foreign protein containing a gene construct which comprises:
(i) a promoter;
(ii) a polynucleotide operably linked to the promoter encoding a polypeptide fragment containing a signal sequence and/or N-region of the leader sequence or variants thereof in which the pI value is controlled; and
(iii) a polynucleotide operably linked to the polynucleotide encoding the polypeptide fragment or variants thereof encoding a secretional enhancer comprising a hydrophilicity enhancing sequence with the controlled pI value.

12. The expression vector according to claim 11, wherein the pI value of the polypeptide fragment containing N-region is controlled to 2.0-4.9 or 5.0-11.0.

13-24. (canceled)

25. A transformant prepared by transforming a host cell with one of the expression vectors of claim 1 or claim 11.

26-28. (canceled)

29. A method for improving secretion efficiency of a foreign protein comprising the following steps:
1) designing a leader sequence having a signal sequence and/or N-region of the leader sequence of a foreign protein with the controlled pI value of 3.09-9.89 or 9.90-11.28;
2) regulating the distance between amino acids which affect the pI value in the leader sequence;
3) constructing a gene construct composed of a polynucleotide encoding a fusion protein containing the leader sequence of step 1), the controlled distance region of step 2), a protease recognition site and the foreign protein in that order;
4) constructing a recombinant expression vector by inserting operably the gene construct of step 3) into a general expression vector;
5) generating a transformant by transforming a host cell with the recombinant expression vector of step 4); and,
6) selecting a transformant from the culture of the transformant of step 5) exhibiting the highest soluble expression of the target protein.

30-33. (canceled)

34. A method for improving secretion efficiency of a foreign protein comprising the following steps:
1) designing a leader sequence having a signal sequence and/or N-region of the leader sequence of a foreign protein with the controlled pI value of 2.0-4.9 or 5.0-11.0;
2) constructing a gene construct composed of a polynucleotide encoding a fusion protein containing the leader sequence of step 1), a hydrophilic secretional enhancer, a protease recognition site and the foreign protein in that order;
3) constructing a recombinant expression vector by inserting the gene construct of step 2) operably into a general expression vector;
4) generating a transformant by transforming a host cell with the recombinant expression vector of step 3); and
5) selecting a transformant from the culture of the transformant of step 4) exhibiting the highest soluble expression of the target protein.

35-39. (canceled)

40. A method for producing a recombinant fusion foreign protein comprising the following steps:
1) designing a leader sequence having a signal sequence and/or N-region of the leader sequence of a foreign protein with the controlled pI value of 3.09-9.89 or 9.90-11.28;
2) regulating the distance between amino acids which affect the pI value in the leader sequence;
3) constructing a gene construct composed of a polynucleotide encoding a fusion protein containing the leader sequence of step 1), the controlled distance region of step 2), a protease recognition site and the foreign protein in that order;
4) constructing a recombinant expression vector by inserting operably the gene construct of step 3) into a general expression vector;
5) generating a transformant by transforming a host cell with the recombinant expression vector of step 4);
6) culturing the transformant of step 5); and
7) separating the recombinant fusion foreign protein from the culture solution of step 6).

41-44. (canceled)

45. A method for producing a recombinant fusion foreign protein comprising the following steps:
1) designing a leader sequence having a signal sequence and/or N-region of the leader sequence of a foreign protein with the controlled pI value of 2.0-4.9 or 5.0-11.0;
2) constructing a gene construct composed of a polynucleotide encoding a fusion protein containing the leader sequence of step 1), a hydrophilic secretional enhancer, a protease recognition site and the foreign protein in that order;
3) constructing a recombinant expression vector by inserting the gene construct of step 2) operably into a general expression vector;
4) generating a transformant by transforming a host cell with the recombinant expression vector of step 3);
5) culturing the transformant of step 4); and
6) separating the recombinant fusion foreign protein from the culture solution of step 5).

46-49. (canceled)

50. A recombinant fusion foreign protein prepared by the method of claim 45.

51. A recombinant fusion foreign protein prepared by the method of claim 40.

52. A pharmaceutical composition containing the recombinant fusion protein of claim 51 and a pharmaceutically acceptable carrier.

53. A method of treating brain disease, comprising administering an effective amount of the pharmaceutical composition of claim 52 to a subject with a brain disease.

54. A method for producing a foreign protein in native form comprising the following steps:
1) designing a leader sequence having a signal sequence and/or N-region of the leader sequence of a foreign protein with the controlled pI value of 3.09-9.89 or 9.90-11.28;
2) regulating the distance between amino acids which affect the pI value in the leader sequence;
3) constructing a gene construct composed of a polynucleotide encoding a fusion protein containing the leader sequence of step 1), the controlled distance region of step 2), a protease recognition site and the foreign protein in that order;
4) constructing a recombinant expression vector by inserting operably the gene construct of step 3) into a general expression vector;
5) generating a transformant by transforming a host cell with the recombinant expression vector of step 4);
6) culturing the transformant of step 5);
7) separating the recombinant fusion foreign protein from the culture solution of step 6); and,
8) separating the foreign protein in native form after cleaving the fusion foreign protein of step 7) with a protease that could cleave the protease recognition site.

55-58. (canceled)

59. A method for producing a foreign protein in native form comprising the following steps:
1) designing a leader sequence having a signal sequence and/or N-region of the leader sequence of a foreign protein with the controlled pI value of 2.0-4.9 or 5.0-11.0;
2) constructing a gene construct composed of a polynucleotide encoding a fusion protein containing the leader sequence of step 1), a hydrophilic secretional enhancer, a protease recognition site and the foreign protein in that order;
3) constructing a recombinant expression vector by inserting the gene construct of step 2) operably into a general expression vector;
4) generating a transformant by transforming a host cell with the recombinant expression vector of step 3);
5) culturing the transformant of step 4);
6) separating the fusion foreign protein from the culture solution of step 5); and,
7) separating the foreign protein in native form after cleaving the fusion foreign protein of step 6) with a protease that could cleave the protease recognition site.

60-64. (canceled)

65. A method for producing an intracellular carrier for the delivery of a target material comprising the following steps:
1) designing a leader sequence having a signal sequence and/or N-region of the leader sequence of a foreign protein with the controlled pI value of 3.09-9.89 or 9.90-11.28;
2) regulating the distance between amino acids which affect the pI value in the leader sequence;
3) constructing a gene construct composed of a polynucleotide encoding a fusion protein containing the leader sequence of step 1), the controlled distance region of step 2), a protease recognition site and the foreign protein in that order;
4) constructing a recombinant expression vector by inserting operably the gene construct of step 3) into a general expression vector;
5) generating a transformant by transforming a host cell with the recombinant expression vector of step 4);
6) culturing the transformant of step 5);
7) separating the fusion foreign protein from the culture solution of step 6);
8) separating the peptide containing the leader sequence, the hydrophilic secretional enhancer and the protease recognition site but not the foreign protein in native form after cleaving the fusion foreign protein of step 7) with a protease that could cleave the protease recognition site; and,
9) combining the peptide containing the leader sequence, the hydrophilic secretional enhancer and the protease recognition site of step 8) with a target material which is supposed to be delivered into the cell.

66-71. (canceled)

72. A method for producing an intracellular carrier for the delivery of a target material comprising the following steps:
1) designing a leader sequence having a signal sequence and/or N-region of the leader sequence of a foreign protein with the controlled pI value of 2.0-4.9 or 5.0-11.0;
2) constructing a gene construct composed of a polynucleotide encoding a fusion protein containing the leader sequence of step 1), a hydrophilic secretional enhancer, a protease recognition site and the foreign protein in that order;
3) constructing a recombinant expression vector by inserting the gene construct of step 2) operably into a general expression vector;
4) generating a transformant by transforming a host cell with the recombinant expression vector of step 3);
5) culturing the transformant of step 4);
6) separating the fusion foreign protein from the culture solution of step 5);
7) separating the peptide containing the leader sequence, the hydrophilic secretional enhancer and the protease recognition site but not the foreign protein in native form after cleaving the fusion foreign protein of step 6) with a protease that could cleave the protease recognition site; and
8) combining the peptide containing the leader sequence, the hydrophilic secretional enhancer and the protease recognition site of step 7) with a target material which is supposed to be delivered into the cell.

73-79. (canceled)


SEQUENCE LISTING (109 sequences)

SEQ ID NO: 1 - DNA, 33 bp, Artificial Sequence
  Synthetic oligonucleotide primer
catatggctaagccgtcttatccgccaacctac

SEQ ID NO: 2 - DNA, 36 bp, Artificial Sequence
  Synthetic oligonucleotide primer
catatgaaagctaagccgtcttatccgccaacctac

SEQ ID NO: 3 - DNA, 36 bp, Artificial Sequence
  Synthetic oligonucleotide primer
catatgaaaaaggctaagccgtcttatccgccaacc

SEQ ID NO: 4 - DNA, 39 bp, Artificial Sequence
  Synthetic oligonucleotide primer
catatgaaaaaaaaagctaagccgtcttatccgccaacc

SEQ ID NO: 5 - DNA, 42 bp, Artificial Sequence
  Synthetic oligonucleotide primer
catatgaaaaaaaaaaaagctaagccgtcttatccgccaacc

SEQ ID NO: 6 - DNA, 45 bp, Artificial Sequence
  Synthetic oligonucleotide primer
catatgaaaaaaaaaaaaaaagctaagccgtcttatccgccaacc

SEQ ID NO: 7 - DNA, 48 bp, Artificial Sequence
  Synthetic oligonucleotide primer
catatgaaaaaaaaaaaaaaaaaagctaagccgtcttatccgccaacc

SEQ ID NO: 8 - DNA, 54 bp, Artificial Sequence
  Synthetic oligonucleotide primer
catatgaaaaaaaaaaaaaaaaaaaaaaaagctaagccgtcttatccgccaacc

SEQ ID NO: 9 - DNA, 36 bp, Artificial Sequence
  Synthetic oligonucleotide primer
catatgcgtgctaagccgtcttatccgccaacctac

SEQ ID NO: 10 - DNA, 36 bp, Artificial Sequence
  Synthetic oligonucleotide primer
catatgcgtcgcgctaagccgtcttatccgccaacc

SEQ ID NO: 11 - DNA, 42 bp, Artificial Sequence
  Synthetic oligonucleotide primer
catatgcgtcgccgtcgcgctaagccgtcttatccgccaacc

SEQ ID NO: 12 - DNA, 48 bp, Artificial Sequence
  Synthetic oligonucleotide primer
catatgcgtcgccgtcgccgtcgcgctaagccgtcttatccgccaacc

SEQ ID NO: 13 - DNA, 54 bp, Artificial Sequence
  Synthetic oligonucleotide primer
catatgcgtcgccgtcgccgtcgccgtcgcgctaagccgtcttatccgccaacc

SEQ ID NO: 14 - DNA, 21 bp, Artificial Sequence
  Synthetic oligonucleotide primer
ctcgaggtcgacaagcttacg

SEQ ID NO: 15 - protein, 3 aa, Artificial Sequence
  Recombinant leader peptide sequence
MAK

SEQ ID NO: 16 - protein, 4 aa, Artificial Sequence
  Recombinant leader peptide sequence
MKAK

SEQ ID NO: 17 - protein, 5 aa, Artificial Sequence
  Recombinant leader peptide sequence
MKKAK

SEQ ID NO: 18 - protein, 6 aa, Artificial Sequence
  Recombinant leader peptide sequence
MKKKAK

SEQ ID NO: 19 - protein, 7 aa, Artificial Sequence
  Recombinant leader peptide sequence
MKKKKAK

SEQ ID NO: 20 - protein, 8 aa, Artificial Sequence
  Recombinant leader peptide sequence
MKKKKKAK

SEQ ID NO: 21 - protein, 9 aa, Artificial Sequence
  Recombinant leader peptide sequence
MKKKKKKAK

SEQ ID NO: 22 - protein, 11 aa, Artificial Sequence
  Recombinant leader peptide sequence
MKKKKKKKKAK

SEQ ID NO: 23 - protein, 4 aa, Artificial Sequence
  Recombinant leader peptide sequence
MRAK

SEQ ID NO: 24 - protein, 5 aa, Artificial Sequence
  Recombinant leader peptide sequence
MRRAK

SEQ ID NO: 25 - protein, 7 aa, Artificial Sequence
  Recombinant leader peptide sequence
MRRRRAK

SEQ ID NO: 26 - protein, 9 aa, Artificial Sequence
  Recombinant leader peptide sequence
MRRRRRRAK

SEQ ID NO: 27 - protein, 11 aa, Artificial Sequence
  Recombinant leader peptide sequence
MRRRRRRRRAK

SEQ ID NO: 28 - DNA, 48 bp, Artificial Sequence
  Synthetic oligonucleotide primer
catatggacgatgacgatgacgctgcaccgtcttatccgccaacctac

SEQ ID NO: 29 - DNA, 42 bp, Artificial Sequence
  Synthetic oligonucleotide primer
catatggacgatgacgctgcaccgtcttatccgccaacctac

SEQ ID NO: 30 - DNA, 33 bp, Artificial Sequence
  Synthetic oligonucleotide primer
catatggaagagccgtcttatccgccaacctac

SEQ ID NO: 31 - DNA, 33 bp, Artificial Sequence
  Synthetic oligonucleotide primer
catatggctgaaccgtcttatccgccaacctac

SEQ ID NO: 32 - DNA, 33 bp, Artificial Sequence
  Synthetic oligonucleotide primer
catatggctgcaccgtcttatccgccaacctac

SEQ ID NO: 33 - DNA, 33 bp, Artificial Sequence
  Synthetic oligonucleotide primer
catatgtgccacccgtcttatccgccaacctac

SEQ ID NO: 34 - DNA, 33 bp, Artificial Sequence
  Synthetic oligonucleotide primer
catatggctcacccgtcttatccgccaacctac

SEQ ID NO: 35 - protein, 8 aa, Artificial Sequence
  Recombinant leader peptide sequence
MDDDDDAA

SEQ ID NO: 36 - protein, 6 aa, Artificial Sequence
  Recombinant leader peptide sequence
MDDDAA

SEQ ID NO: 37 - protein, 3 aa, Artificial Sequence
  Recombinant leader peptide sequence
MEE

SEQ ID NO: 38 - protein, 3 aa, Artificial Sequence
  Recombinant leader peptide sequence
MAE

SEQ ID NO: 39 - protein, 3 aa, Artificial Sequence
  Recombinant leader peptide sequence
MAA

SEQ ID NO: 40 - protein, 3 aa, Artificial Sequence
  Recombinant leader peptide sequence
MCH

SEQ ID NO: 41 - protein, 3 aa, Artificial Sequence
  Recombinant leader peptide sequence
MAH

SEQ ID NO: 42 - DNA, 51 bp, Artificial Sequence
  Synthetic oligonucleotide primer
catatggaagagatcgaaggtcgtgctaagccgtcttatccgccaacctac

SEQ ID NO: 43 - DNA, 57 bp, Artificial Sequence
  Synthetic oligonucleotide primer
catatggaagagccgtctatcgaaggtcgtgctaagccgtcttatccgccaacctac

SEQ ID NO: 44 - DNA, 63 bp, Artificial Sequence
  Synthetic oligonucleotide primer
catatggaagagccgtcttatccgatcgaaggtcgtgctaagccgtcttatccgccaacctac

SEQ ID NO: 45 - DNA, 69 bp, Artificial Sequence
  Synthetic oligonucleotide primer
catatggaagagccgtcttatccgccaaccatcgaaggtcgtgctaagccgtcttatccgccaacctac

SEQ ID NO: 46 - protein, 7 aa, Artificial Sequence
  Recombinant leader peptide sequence containing Xa recognition site
MEEIEGR

SEQ ID NO: 47 - protein, 9 aa, Artificial Sequence
  Recombinant leader peptide sequence containing Xa recognition site
MEEPSIEGR

SEQ ID NO: 48 - protein, 11 aa, Artificial Sequence
  Recombinant leader peptide sequence containing Xa recognition site
MEEPSYPIEGR

SEQ ID NO: 49 - protein, 13 aa, Artificial Sequence
  Recombinant leader peptide sequence containing Xa recognition site
MEEPSYPPTIEGR

SEQ ID NO: 50 - DNA, 63 bp, Artificial Sequence
  Synthetic oligonucleotide primer
catatgaaaaagacagctatcgcgattgcagtggcagctaagccgtcttatccgccaacctac

SEQ ID NO: 51 - DNA, 63 bp, Artificial Sequence
  Synthetic oligonucleotide primer
catatgaaaaagacagctatcgcgattgcagtggcagctgcaccgtcttatccgccaacctac

SEQ ID NO: 52 - DNA, 63 bp, Artificial Sequence
  Synthetic oligonucleotide primer
catatgaaagctacaaagatcgcgattgcagtggcagctgcaccgtcttatccgccaacctac

SEQ ID NO: 53 - DNA, 63 bp, Artificial Sequence
  Synthetic oligonucleotide primer
catatgaaagctacagctatcaagattgcagtggcagctgcaccgtcttatccgccaacctac

SEQ ID NO: 54 - DNA, 63 bp, Artificial Sequence
  Synthetic oligonucleotide primer
catatgaaagctacagctatcgcgattaaggtggcagctgcaccgtcttatccgccaacctac

SEQ ID NO: 55 - DNA, 63 bp, Artificial Sequence
  Synthetic oligonucleotide primer
catatgaaagctacagctatcgcgattgcagtgaaggctgcaccgtcttatccgccaacctac

SEQ ID NO: 56 - protein, 13 aa, Artificial Sequence
  Synthetic oligonucleotide primer
MKKTAIAIAVAAK

SEQ ID NO: 57 - protein, 13 aa, Artificial Sequence
  Recombinant leader peptide sequence
MKKTAIAIAVAAA

SEQ ID NO: 58 - protein, 13 aa, Artificial Sequence
  Recombinant leader peptide sequence
MKATKIAIAVAAA

SEQ ID NO: 59 - protein, 13 aa, Artificial Sequence
  Recombinant leader peptide sequence
MKATAIKIAVAAA

SEQ ID NO: 60 - protein, 13 aa, Artificial Sequence
  Recombinant leader peptide sequence
MKATAIAIKVAAA

SEQ ID NO: 61 - protein, 13 aa, Artificial Sequence
  Recombinant leader peptide sequence
MKATAIAIAVKAA

SEQ ID NO: 62 - DNA, 51 bp, Artificial Sequence
  Synthetic oligonucleotide primer
catatgcgtcgccgtcgccgtcgccgtcacatcagccacatctccatgtgc

SEQ ID NO: 63 - DNA, 51 bp, Artificial Sequence
  Synthetic oligonucleotide primer
catatgaaaaaaaaaaaaaaaaaaaaacacatcagccacatctccatgtgc

SEQ ID NO: 64 - DNA, 51 bp, Artificial Sequence
  Synthetic oligonucleotide primer
catatgcaccatcaccatcaccatcaccacatcagccacatctccatgtgc

SEQ ID NO: 65 - DNA, 51 bp, Artificial Sequence
  Synthetic oligonucleotide primer
catatgtactattactattactattaccacatcagccacatctccatgtgc

SEQ ID NO: 66 - DNA, 51 bp, Artificial Sequence
  Synthetic oligonucleotide primer
catatgtgctgttgctgttgctgttgccacatcagccacatctccatgtgc

SEQ ID NO: 67 - DNA, 51 bp, Artificial Sequence
  Synthetic oligonucleotide primer
catatggaagaggaagaggaagaggaacacatcagccacatctccatgtgc

SEQ ID NO: 68 - DNA, 51 bp, Artificial Sequence
  Synthetic oligonucleotide primer
catatggacgatgacgatgacgatgaccacatcagccacatctccatgtgc

SEQ ID NO: 69 - DNA, 45 bp, Artificial Sequence
  Synthetic oligonucleotide primer
ctcgaggtcgacaagcttttcgaacttgcagcaggggccacagcc

SEQ ID NO: 70 - protein, 8 aa, Artificial Sequence
  Recombinant leader peptide sequence
MRRRRRRR

SEQ ID NO: 71 - protein, 8 aa, Artificial Sequence
  Recombinant leader peptide sequence
MKKKKKKK

SEQ ID NO: 72 - protein, 8 aa, Artificial Sequence
  Recombinant leader peptide sequence
MHHHHHHH

SEQ ID NO: 73 - protein, 8 aa, Artificial Sequence
  Recombinant leader peptide sequence
MYYYYYYY

SEQ ID NO: 74 - protein, 8 aa, Artificial Sequence
  Recombinant leader peptide sequence
MCCCCCCC

SEQ ID NO: 75 - protein, 8 aa, Artificial Sequence
  Recombinant leader peptide sequence
MEEEEEEE

SEQ ID NO: 76 - protein, 8 aa, Artificial Sequence
  Recombinant leader peptide sequence
MDDDDDDD

SEQ ID NO: 77 - DNA, 87 bp, Artificial Sequence
  Synthetic oligonuceotide primer
catatggctcacacagctatcgcgattgcagtgcgccgtcgccgtcgccgtatcgaaggtcgtcacatcagccacatctccatgtgc

SEQ ID NO: 78 - DNA, 87 bp, Artificial Sequence
  Synthetic oligonuceotide primer
catatggctcacacagctatcgcgattgcagtgtactattactattactatatcgaaggtcgtcacatcagccacatctccatgtgc

SEQ ID NO: 79 - DNA, 87 bp, Artificial Sequence
  Synthetic oligonuceotide primer
catatggctcacacagctatcgcgattgcagtggaagaggaagaggaagagatcgaaggtcgtcacatcagccacatctccatgtgc

SEQ ID NO: 80 - DNA, 87 bp, Artificial Sequence
  Synthetic oligonuceotide primer
catatggctgcaacagctatcgcgattgcagtgcgccgtcgccgtcgccgtatcgaaggtcgtcacatcagccacatctccatgtgc

SEQ ID NO: 81 - DNA, 87 bp, Artificial Sequence
  Synthetic oligonuceotide primer
catatggctgcaacagctatcgcgattgcagtgtactattactattactatatcgaaggtcgtcacatcagccacatctccatgtgc

SEQ ID NO: 82 - DNA, 87 bp, Artificial Sequence
  Synthetic oligonuceotide primer
catatggctgcaacagctatcgcgattgcagtggaagaggaagaggaagagatcgaaggtcgtcacatcagccacatctccatgtgc

SEQ ID NO: 83 - DNA, 87 bp, Artificial Sequence
  Synthetic oligonuceotide primer
catatggaagagacagctatcgcgattgcagtgcgccgtcgccgtcgccgtatcgaaggtcgtcacatcagccacatctccatgtgc

SEQ ID NO: 84 - DNA, 87 bp, Artificial Sequence
  Synthetic oligonuceotide primer
catatggaagagacagctatcgcgattgcagtgtactattactattactatatcgaaggtcgtcacatcagccacatctccatgtgc

SEQ ID NO: 85 - DNA, 87 bp, Artificial Sequence
  Synthetic oligonuceotide primer
catatggaagagacagctatcgcgattgcagtggaagaggaagaggaagagatcgaaggtcgtcacatcagccacatctccatgtgc

SEQ ID NO: 86 - protein, 20 aa, Artificial Sequence
  Signal sequence with the low pI value + secretional enhancer sequence
MAHTAIAIAVRRRRRRIEGR

SEQ ID NO: 87 - protein, 20 aa, Artificial Sequence
  Signal sequence with the low pI value + secretional enhancer sequence
MAHTAIAIAVYYYYYYIEGR

SEQ ID NO: 88 - protein, 20 aa, Artificial Sequence
  Signal sequence with the low pI value + secretional enhancer sequence
MAHTAIAIAVEEEEEEIEGR

SEQ ID NO: 89 - protein, 20 aa, Artificial Sequence
  Signal sequence with the low pI value + secretional enhancer sequence
MAATAIAIAVRRRRRRIEGR

SEQ ID NO: 90 - protein, 20 aa, Artificial Sequence
  Signal sequence with the low pI value + secretional enhancer sequence
MAATAIAIAVYYYYYYIEGR

SEQ ID NO: 91 - protein, 20 aa, Artificial Sequence
  Signal sequence with the low pI value + secretional enhancer sequence
MAATAIAIAVEEEEEEIEGR

SEQ ID NO: 92 - protein, 20 aa, Artificial Sequence
  Signal sequence with the low pI value + secretional enhancer sequence
MEETAIAIAVRRRRRRIEGR

SEQ ID NO: 93 - protein, 20 aa, Artificial Sequence
  Signal sequence with the low pI value + secretional enhancer sequence
MEETAIAIAVYYYYYYIEGR

SEQ ID NO: 94 - protein, 20 aa, Artificial Sequence
  Signal sequence with the low pI value + secretional enhancer sequence
MEETAIAIAVEEEEEEIEGR

SEQ ID NO: 95 - protein, 10 aa, Artificial Sequence
  The repeating decapeptide unit of Mytilus edulis foot protein (1Mefp1)
AKPSYPPTYK

SEQ ID NO: 96 - DNA, 30 bp, Artificial Sequence
  Synthetic oligonucleotide primer for mefp1
tacaaagctaagccgtcttatccgccaacc

SEQ ID NO: 97 - DNA, 30 bp, Artificial Sequence
  Synthetic oligonucleotide primer for mefp1
tttgtaggttggcggataagacggcttagc

SEQ ID NO: 98 - DNA, 17 bp, Artificial Sequence
  Synthetic oligonucleotide primer for La
gatccgaattccccggg

SEQ ID NO: 99 - DNA, 19 bp, Artificial Sequence
  Synthetic oligonucleotide primer for La
tttgtacccggggaattcg

SEQ ID NO: 100 - DNA, 22 bp, Artificial Sequence
  Synthetic oligonucleotide primer for Ra
tacaaacgtaagcttgtcgacc

SEQ ID NO: 101 - DNA, 20 bp, Artificial Sequence
  Synthetic oligonucleotide primer for Ra
tcgaggtcgacaagcttacg

SEQ ID NO: 102 - DNA, 51 bp, Artificial Sequence
  Synthetic oligonucleotide primer
catatgaaagctacagctatcaaggctaagccgtcttatccgccaacctac

SEQ ID NO: 103 - DNA, 54 bp, Artificial Sequence
  Synthetic oligonucleotide primer
catatgaaagctacagctatcaaggcagctaagccgtcttatccgccaacctac

SEQ ID NO: 104 - DNA, 57 bp, Artificial Sequence
  Synthetic oligonucleotide primer
catatgaaagctacagctatcaaggctgcagctaagccgtcttatccgccaacctac

SEQ ID NO: 105 - DNA, 60 bp, Artificial Sequence
  Synthetic oligonucleotide primer
catatgaaagctacagctatcaaggcagctgcagctaagccgtcttatccgccaacctac

SEQ ID NO: 106 - protein, 9 aa, Artificial Sequence
  Recombinant leader peptide sequence
MKATAIKAK

SEQ ID NO: 107 - protein, 10 aa, Artificial Sequence
  Recombinant leader peptide sequence
MKATAIKAAK

SEQ ID NO: 108 - protein, 11 aa, Artificial Sequence
  Recombinant leader peptide sequence
MKATAIKAAAK

SEQ ID NO: 109 - protein, 12 aa, Artificial Sequence
  Recombinant leader peptide sequence
MKATAIKAAAAK